(19) United States
(12) Patent Application Publication
(10) Pub. No.: US 2007/0129634 A1
(43) Pub. Date: Jun. 7, 2007

(54) Biomedical positioning and stabilization system

(76) Inventors: Katherine M. Hickey, Enfield, NH (US); Amish Parashar, Rossmoor, CA (US); Brian D. Sites, Plainfield, NH (US); Brian Conley Spence, Orford, NH (US)

Correspondence Address: HINCKLEY, ALLEN & SNYDER, LLP, 43 NORTH MAIN STREET, CONCORD, NH, 03301, US

(21) Appl. No.: 11/338,270

(22) Filed: Jan. 23, 2006

Related U.S. Application Data
(60) Provisional application No. 60/727,319, filed on Oct. 17, 2005.

(51) Int. Cl. A61B 8/00; A45D 19/04; E04G 3/00; A47J 47/16; F16D 3/00; F16M 11/00; F16D 1/00; F16D 1/12; F16C 11/00; F16C 11/06; A61B 19/00

(52) U.S. Cl. 600/439; 403/24; 403/91; 606/130; 600/461; 248/274.1; 248/127; 248/451

(57) ABSTRACT

This invention provides a support device that allows the adjustable, yet rigid placement of a probe or other medical instrument against a region of interest/treatment on a patient. The system and method of rigid fixation, positioning, and adjustment contemplated herein is useful for a broad array of medical procedures including, but not limited to, ultrasound-guided anesthetic delivery. In an exemplary embodiment of the present invention, a flexible armature is attached to a rigid stand placed upon the floor, or attached to another stable surface such as a bed rail, wall, ceiling or piece of equipment. A joint connects the armature to an instrument holder able to accommodate and rigidly attach an ultrasound sensing probe or other medical device. The medical device then remains rigidly attached to the described invention during the procedure. Furthermore, this set position is resistant to minor patient motion or other disturbances. If required, small alterations can be made by the operator during the procedure with minimal effort. Such adjustment may be desirable, for example, if access to a new anatomical structure is needed. In this manner, the primary operator is able to maintain a ‘hands-free’ approach.

### CROSS-REFERENCE TO RELATED APPLICATION

The present application claims the benefit of U.S. Provisional Patent Application Ser. No. 60/727,319, which was filed on Oct. 17, 2005, by Brian D. Sites, et al. for a BIOMEDICAL POSITIONING AND STABILIZATION SYSTEM and is hereby incorporated by reference.

### BACKGROUND OF THE INVENTION

1. Field of the Invention

The invention relates to medical devices and, in particular, to a device for positioning and stabilizing diagnostic or therapeutic devices used in medical procedures.

2. Background Information

In clinical practice, there are many different procedures utilized for various diagnostic, therapeutic, or monitoring applications. These are typically conducted by a highly skilled operator who relies heavily on the ability to simultaneously perform multiple tasks, such as viewing a monitor while positioning a probe or dissecting tissue while exerting separation force upon the walls of an incision. Examples of such medical tasks include, but are not limited to, invasive radiology (for breast biopsy), local anesthesia (for peripheral nerve blocks), invasive cardiology (for stent placement and deployment), vascular surgery (for measuring intravascular blood flow), and general surgery (for retraction of the incision walls or holding a hemostat clamping device).

For example, hemostatic clamping devices, commonly referred to as “hemostats”, are used by surgeons to temporarily occlude blood flow through a vessel. This may be useful in the course of surgery in order to prevent the loss of blood or maintain an operating area. Often, these devices are held by an assisting operator, but they may also be left unsupported. When used, the skilled operator is required to hold and stabilize a single instrument for a period of time while the surgical procedure proceeds. At the end of the required time, the clamp is released to restore flow through the targeted vessel.

By way of a second example, a vascular surgeon may clamp a blood vessel with a flow probe in order to quantify volumetric flow through the vessel. The flow probe is placed and secured around the target vessel and then connected to the appropriate measurement equipment. In order to gather accurate measurements, the operator must hold the probe so as to maintain a specific orientation to the target vessel (perpendicular to the axis of flow). The operator is therefore employed solely to support the measurement device in a given orientation, and is therefore unable to contribute to the major goal of the surgical procedure while the measurement is underway.

By way of a third example, peripheral nerve blocks are used by anesthesiologists and pain doctors to anesthetize nerves that are involved in the transmission of pain signals during surgery or states of chronic disease. The image-acquisition procedure requires at least one of the operator's hands to be continually occupied at a high level of concentration and dexterity for probe manipulation. In general, the practitioner must carefully orient the probe and maintain it in relative contact with the anatomical region to acquire a good image. A second skilled operator must be employed to insert the needle, then deliver the required drug; undoubtedly a task requiring two hands.

Peripheral nerve blocks fall under the category of regional anesthesia, which indicates only a portion of the body is anesthetized and/or desensitized. This is in contrast to general anesthesia, in which the patient is placed into a state of complete unconsciousness. Nerve blocks entail the deposition of local anesthetics, such as lidocaine, which block the transmission of the pain signals for a variable amount of time. The major challenge for the clinician performing nerve blocks is related to finding the nerve of interest. Traditional anesthesiology approaches rely on palpating external landmarks on the skin, assuming that the anatomy below is normal, and subsequently inserting a needle attached to a nerve stimulator. When the needle contacts the nerve, a twitch occurs in the muscle that is interconnected with the nerve. By this method, the practitioner knows where to inject.

Because anatomy is variable, this technique results in significant failure rates, multiple needle passes, and significant potential for pain and injury to the nerve and adjacent structures. Modern ultrasound technologies allow the operator to guide his or her needle under live visualization to the structures of interest. The operator can then avoid multiple needle sticks, avoid structures (such as blood vessels), and confirm that the local anesthetic is spreading around the nerve of interest. However, current ultrasound approaches to performing nerve blocks and any other procedures (placement of intravenous catheters, breast biopsies, etc.) require that the operator hold the ultrasound probe in at least one hand in engagement with the anatomical region of interest. Once a satisfactory image of the structure is acquired, subtle movements of the hand holding the probe may result in degradation of the image, requiring a repositioning of the probe.

The pressure applied to the region of interest/treatment by the procedure guiding probe is critical. Too much pressure tends to distort the underlying tissues, making for an inaccurate image and pinching of internal tissues that may lead to misdirection of the is needle. Too little pressure yields a bad image. During the procedure, the guiding probe is employed previous to needle insertion. Hence, the practitioner generally uses the “strong” hand (e.g. the right hand for a right-handed person) to guide and position the probe. This leaves the task of needle insertion either to the practitioner's weak hand or a second practitioner. The single-practitioner approach is rarely used in practice, both due to quality and safety concerns and also to prevailing medical practice rules and custom. Hence, two practitioners are, in fact, employed to perform the procedure (e.g. block, placement of intravenous catheters, breast biopsies). The second practitioner is needed to hold the probe, as the primary operator administers therapy—a task encompassing the injection of medicine, placement of the catheter, or performing the biopsy.

As a specific example, FIG. 1 illustrates the current methodology for regional anesthesiology needle-placement under ultrasound visualization. The skin area 115 of the patient 100 is exposed and sterilized. The needle 120 is inserted through the skin 115 while an ultrasound probe 130 is held atop skin 115. In this manner, visualization of the underlying anatomy is established by operator viewing of the ultrasound monitor/imaging system (not shown) attached to probe 130 by probe cable 135. As the needle 120 is inserted through skin 115, visualization is continually maintained by use of the ultrasound system. As taught today, the operator's non-dominant hand 140 should maintain control of the ultrasound probe while the dominant hand 145 guides the needle 120 into the desired neural or other anatomy. A second skilled operator is required for drug delivery. This approach necessarily requires three operator hands for needle 120 placement, and probe 130 manipulation, and drug delivery. Since this approach is mainly to deliver anesthetic, there may also be one or more additional operators (specialists/surgeons) present who subsequently performs the primary operation with properly prepared tools.

Note that the probe 130 or any other device contacting the patient or an operator's hands/equipment must be sterile. This is accomplished by applying a sterile drape (typically clear sterile plastic sheeting, such as polyethylene (often arranged in a bag-like configuration), over the device. This process of covering device with a sterile “bag” adds further complexity as the practitioner is wearing sterile gloves that can become contaminated by the process of applying the sterile bag. Thus, even this simple process requires further assistance that entails additional personnel, and hence, cost greater for the procedure.

It follows that a device capable of providing operating surgeons or other clinicians with the use of both hands during a medical procedure would be useful in a variety of situations. Such a device would also be useful because it would reduce the amount of corrective repositioning a device, imaging or otherwise, must undergo due to clinician movement. Setup time can be greatly reduced and the presence of secondary clinicians eliminated with a device that functions as a reliable third clinician “hand.”

Examples of devices used to create hands-free adjustable environments in non-medical fields are the microphone stand, e.g. U.S. Pat. No. 5,340,066, the headlamp, e.g. U.S. Pat. No. 4,462,064, the indicator holder, e.g. U.S. Pat. No. 5,704,132, and the ergonomic keyboard adjustment system, e.g. U.S. Pat. No. 5,257,767. Each of these devices provides the operator with a means by which they may hold or control a device in a steady and reliable manner without the loss of the use of either hand.

In the medical field, an additional layer of complexity is added to the development of devices that create a hands-free environment, due to anatomical considerations, which differ from patient to patient, to clinician preference regarding the positioning and use of equipment, and the above-described need to maintain a sterile environment, which dictates the use of certain materials and form factors. Furthermore, as the range of medical devices that may be enhanced by a rigid support system is broad, it follows that armature positioning requirements will vary dramatically from one usage category to another.

General-use devices which are easily employable in a variety of medical settings to provide operators with hands-free environments are not frequently disclosed. In one example, U.S. Pat. No. 5,626,322 discloses a simple pivoting rod system used to hold rigid a generic medical portable device throughout the duration of a task. In another example, U.S. Pat. Nos. 5,284,313 and 6,138,970 teach devices employing a telescoping and pivoting rod system. Similarly, U.S. Pat. No. 5,671,900 discloses a rigid stand system, encompassing a telescopic rod assembly, a stand and a clamp to hold the necessary device. Nevertheless, each of these devices provides only limited user maneuverability and fine adjustment. They do not provide for user manipulation in more than two planes/degrees of freedom, and/or do not provide for rotational motion about one or more axes at the distal end—a significant limitation in a surgical environment. Furthermore, operation of these rigid, and often heavy, support systems is cumbersome, frequently requiring the use of more than two hands to properly position them without incurring damage to the article used or person undergoing a medical procedure. U.S. Patent Publication No. 2002/014567 does disclose a multi-jointed armature support system with fluid locks, which features eliminate the difficulty associated with positioning and locking the multi-segmented arms of the above-described devices, but retains many other of the noted disadvantages.

Most current devices used to create hands-free environments in the medical field have been developed for very specific procedures. An example is the cardiac surgery stabilization device “Octopus” disclosed in U.S. Pat. No. 6,464,630. This tissue stabilization device uses suction to hold a portion of the heart still to provide a skilled operator with access to a target coronary during beating heart surgery. With the “Octopus” in place, the medical professional has full use of both hands to perform tasks necessary to successful completion of the operation. However, the suction technology used to hold the target anatomy at a desired location is not easily transferred to the task of rigidly positioning surgical tools, including ultrasound probes or surgical retractors. In some cases, the weight, size, or shape of the device to be held may not be accommodated by the prior devices. In others, the level of rigidity and maneuverability of the armature system may not be sufficient.

Assemblies and arrangements where the rigidly held device is not the primary surgical tool or target of surgical intervention (but rather a subsidiary device, like an imaging probe) are less frequently disclosed. For example, U.S. Pat. Nos. 5,170,790 and 6,248,101 each disclose pivoting rod armature systems used to rigidly support exploration probes and imaging devices respectively. These arrangements are cumbersome and difficult to operate quickly and efficiently when only one operator is involved. Furthermore, these arrangements do not provide the ability for the user or operator to create fine adjustments after the device is locked into place. U.S. Pat. Pub. No. 2004/133105 discloses an automated armature system used to rigidly support an imaging device. This device makes single-handed operation of the medical device possible, but may not be responsive to minute changes in the system requiring readjustment of the distal end of the armature. Furthermore, the interface portion of the system may be difficult for some users to operate effectively, mimicking fine adjustments normally made at the hand of a skilled operator, or in a timely manner, without an appreciable learning curve. U.S. Pat. No. 4,963,903 discloses a device used to rigidly hold a camera system at the distal end of a flexible armature configuration. This device encompasses an armature system that is easy to use and accommodates fine adjustments from the user at any stage during the procedure for which it is being used, but it is limited in that it provides no mechanism for rigidly locking the armature into place, relying solely on the stiffness of the flexible arm for locking, and in its scope, being only applicable to camera-like devices.

It is highly desirable, therefore, to provide a holding mechanism or device that facilitates stable positioning and manipulation of a probe or other tool relative to a patient in order to reliably enable procedures, such as hands-free ultrasound-guided intervention. In the case of ultrasound-guided anesthesia, the hands-free environment facilitated by the supporting mechanism should reduce the amount of time spent in a procedure by eliminating both the need for device repositioning after the clinician inadvertently moves the guiding hand, and the problem of the probe slipping over the portion of the patient's anatomy to which it is directed. It also enables the practitioner to perform the operation with both hands and without the need of a second assisting practitioner to maintain orientation of the probe or other tool. Additionally, the holding mechanism should allow substantially continuous acquisition of a fixed image of the anatomical region of interest, thereby enhancing the overall effectiveness and safety of the procedure. To achieve these goals, the holding mechanism should generally allow a probe or other tool to be applied against the patient with a consistent and acceptable force that maintains needed pressurable contact with the region but does not overly compress internal tissues in the region. The holding mechanism should also facilitate maintenance of a sterile environment and allow the quick and easy application of sterile drapes to the holding mechanism and any underlying probe or tool attached thereto, without requiring the practitioner or an assistant to contaminate sterilized hands or tools in the process.

### SUMMARY OF THE INVENTION

This invention overcomes the disadvantages of the prior art by providing a support device that allows the adjustable, yet rigid placement of a probe or other medical instrument against a region of interest/treatment on a patient. The system and method of rigid fixation, positioning, and adjustment contemplated herein is useful for a broad array of medical procedures including, but not limited to, ultrasound-guided anesthetic delivery.

In an exemplary embodiment of the present invention, a flexible armature is attached to a rigid stand placed upon the floor, or attached to another stable surface such as a bed rail, wall, ceiling or piece of equipment. A joint connects the armature to an instrument holder able to accommodate and rigidly attach an ultrasound sensing probe or other medical device. The medical device then remains rigidly attached to the described invention during the procedure. Furthermore, this set position is resistant to minor patient motion or other disturbances. If required, small alterations can be made by the operator during the procedure with minimal effort. Such adjustment may be desirable, for example, if access to a new anatomical structure is needed. In this manner, the primary operator is able to maintain a ‘hands-free’ approach.

In an illustrative embodiment, the armature section is constructed from discrete polymer segments each defining an inner lumen and interengaging hemispherical nose sections and tail sections. The hemispherical nature of the connection affords a degree of bending between segments in three dimensions as well as axial rotation between segments. In a length of approximately 6 inches to 4 feet, the armature is capable of great flexibility, allowing it to be repositioned at will against the patient's region of interest/treatment. The armature can be locked, once positioned by, use of mechanical, electrical or fluid (vacuum) mechanisms that increase segment-to-segment friction or otherwise fix the segments at their current orientation. The lumen of the armature can be provided with a conduit to allow internal routing of electronic and other leads from the probe/distal end. A ball joint can be provided at the proximal end of the armature that allows a degree of flotation to enable the armature to be lifted above the level of a recumbent patient to allow access to the probe, and to enable a moderate degree of weight-generated pressure to be applied to the region of the patient to ensure the probe tip remains effectively (but not distortingly) engaged against the region.

The holder can define a variety of structures some of which allow a wide variety of probe shapes and sizes to be removably engaged. One embodiment defines a quick-disconnect assembly with a connector formed directly on the probe and a receiving connector attached to the distal end of the armature. Another embodiment employs a clamping arrangement to secure the probe. Another embodiment employs a resilient material (such as memory foam) or inflated bladder to frictionally secure the probe. Another embodiment allows remote machine operation via controls located on the holder.

In a method for employing the support device, the practitioner (potentially a single individual) prepares the probe (an ultrasound probe in one example) and/or holder, which interconnects to a display device within view by orienting the armature near or over a recumbent patient. The practitioner can use one sterilized hand to now drape the probe and armature section with a sterile drape that is opened at a proximal end to slide over the armature and sealed at a distal end placed in engagement with a tip of the probe. At no time does the practitioner contact a non-sterile object. The practitioner now uses the sterile hand(s) to reorient the tip of the ultrasound probe into engagement with the skin region of the patient by flexing the armature to overcome predetermined friction between the interengaged nose sections and tail section of at least some of the segments to bend and rotate the segments so as to acquire a desired image at the display device. The armature section maintains a predetermined reoriented shape at least by the predetermined friction. This can be supplemented by activating a locking mechanism on the support device. The practitioner now guides a needle or other instrument to a subcutaneous target with at least one hand while viewing the image and while the ultrasound probe. During this guiding procedure, the instrument holder remains ungrasped by another hand, allowing the practitioner to maintain all focus on the guiding step while an image is maintained. If adjustment of the probe is needed, it is easily reoriented by grasping the holder, flexing and rotating the armature as needed and ungrasping the holder once proper orientation is reestablished. The guiding procedure, or another procedure can then continue with all attention focused thereon.

### BRIEF DESCRIPTION OF THE DRAWINGS

The invention description below refers to the accompanying drawings, of which:

FIG. 1, already described, is an illustration of an operator performing an unsupported ultrasound-guided needle placement procedure according to the prior art;

FIG. 2 is an exploded perspective view of an instrument support device, including various subsystems thereof, and featuring a gooseneck-style segmented armature subsystem, according to an embodiment of this invention;

FIG. 2A is a perspective view of an armature subsystem for the support device composed of a plurality of jointed rods according to an alternate embodiment;

FIG. 2B is a partially exposed top view of an exemplary joint section for the armature subsystem of FIG. 2A;

FIG. 3 is an illustration of an embodiment of the present invention being used as an ultrasound probe stand relative to a region of interest on a patient;

FIG. 4 is an illustration of an embodiment of the present invention being used to grasp a surgical retractor over an open incision on an area of treatment of a patient;

FIG. 5 is a partially exposed side view the support device mounted on a freestanding stand post according to an illustrative embodiment of the invention;

FIG. 6 is a perspective view of the support device of FIG. 5 with protective sleeve removed to reveal the details of the segmented, flexible armature section;

FIG. 7 is a perspective view of the support device according to FIG. 6 with the protective sleeve in place;

FIG. 8 is a broken side cross section of the flexible armature section and probe holder for the support device of FIG. 5;

FIG. 9 is a broken side cross section of the flexible armature section and probe holder for the support device of FIG. 5 including a tension-cable-based armature locking mechanism according to an embodiment of the invention;

FIG. 10 is a cross section of the armature section taken along line 10-10 of FIG. 9, detailing a cable-supporting disk within the interior of a segment;

FIG. 10A is a fragmentary side cross section of the probe holder and attached distal end of the armature section of FIG. 9 further detailing the holder-actuated cable tensioner according to an embodiment of the invention;

FIG. 11 is a broken side cross section of the flexible armature section and probe holder for the support device of FIG. 5 including a vacuum-based armature locking mechanism according to an embodiment of the invention;

FIG. 11A is a cross section of the flexible armature section and probe holder for the support device of FIG. 5 including a hoop-stress-based locking mechanism according to an embodiment of the invention;

FIG. 12 is a fragmentary perspective view showing the construction and motion of a ball joint assembly at the proximal end of the armature section for the support device of FIG. 5;

FIG. 13 is a fragmentary side view showing of the ball joint assembly of FIG. 12 detailing relative motion and rotational lock actuation;

FIG. 14 is a fragmentary cross section of the ball joint assembly of FIG. 12 further detailing a rotational locking mechanism according to an embodiment of the invention;

FIG. 15 is a side cross section of a support device including an L-shaped mounting elbow/base typically for use in implementations in which the support device is used over a recumbent patient;

FIG. 16 is a perspective view of a probe holder including a screw-locked probe clamping mechanism according to an embodiment of the invention;

FIG. 17 is a perspective view of a bed-based mounting arrangement for the support device according to an embodiment of this invention;

FIG. 18 is a perspective view of an IV-stand-based mounting arrangement for the support device according to an embodiment of this invention;

FIG. 19 is a perspective view of a ceiling-based mounting arrangement for the support device according to an embodiment of this invention;

FIG. 20 is a perspective view of a wall/vertical surface-based mounting arrangement for the support device according to an embodiment of this invention;

FIG. 21 is a perspective view of an equipment-housing-based mounting arrangement for the support device according to an embodiment of this invention;

FIG. 22 is a perspective view of a procedure for employing the support device to engage a probe in connection with the application an anesthetic needle to a region of interest/treatment detailing an initial site-preparation step;

FIG. 23 is a perspective view of the procedure for employing the support device to is engage a probe in connection with the application an anesthetic needle to the patient's region of interest/treatment detailing the beginning of a step of sterile draping of the probe and support device;

FIG. 24 is a perspective view of the procedure for employing the support device to engage a probe in connection with the application an anesthetic needle to a region of interest/treatment further detailing the step of draping of the probe and support device;

FIG. 25 is a perspective view of the procedure for employing the support device to engage a probe in connection with the application an anesthetic needle to a region of interest/treatment detailing the completion of the step of draping of the probe and support device;

FIG. 26 is a perspective view of the procedure for employing the support device to engage a probe in connection with the application an anesthetic needle to a region of interest/treatment detailing the step of securing the drape about the probe with an elastic band after reorienting the support device;

FIG. 27 is a perspective view of the procedure for employing the support device to engage a probe in connection with the application an anesthetic needle to a region of interest/treatment detailing the step of applying a drape to the patient's region of interest/treatment;

FIG. 28 is a perspective view of the procedure for employing the support device to engage a probe in connection with the application an anesthetic needle to a region of interest/treatment detailing the step of preparing the anesthetic needle for insertion at the region of interest/treatment;

FIG. 29 is a perspective view of the procedure for employing the support device to engage a probe in connection with the application an anesthetic needle to a region of interest/treatment detailing the step of orienting and engaging the probe tip at the region of interest so as to assist in guiding the needle to the target;

FIG. 29A is a perspective view of the procedure for employing the support device to engage a probe in connection with the application an anesthetic needle to a region of interest/treatment detailing the step of reorienting the support device to apply weight-generated predetermined pressure to the region of interest;

FIG. 30 is a fragmentary side view of the draped holder and probe showing attachment of a needle guide to the probe;

FIG. 31 is a perspective view of the procedure for employing the support device to engage a probe in connection with the application an anesthetic needle to a region of interest/treatment detailing the step of guiding the needle while viewing the progress on a display interconnected to the engaged probe;

FIG. 32 is a perspective view of a procedure for rotating and/or reorienting the probe to acquire a desired image;

FIG. 33 is a perspective view of a procedure for inserting or reinserting a needle through a needle guide attached to the reoriented probe of FIG. 32;

FIG. 34 is a perspective view of a system for quick-connect/disconnect of a probe with respect to a distal support device holder/mounting according to an embodiment of the invention;

FIG. 35 is a perspective view of a system for quick-connect/disconnect of a probe with respect to a distal support device holder/mounting according to another embodiment of the invention, featuring a complex polygonal interconnect shape; and

FIG. 36 is a perspective view of a system for quick-connect/disconnect of a probe with respect to a distal support device holder/mounting according to an embodiment of the invention, featuring an axially aligned interconnect structure.

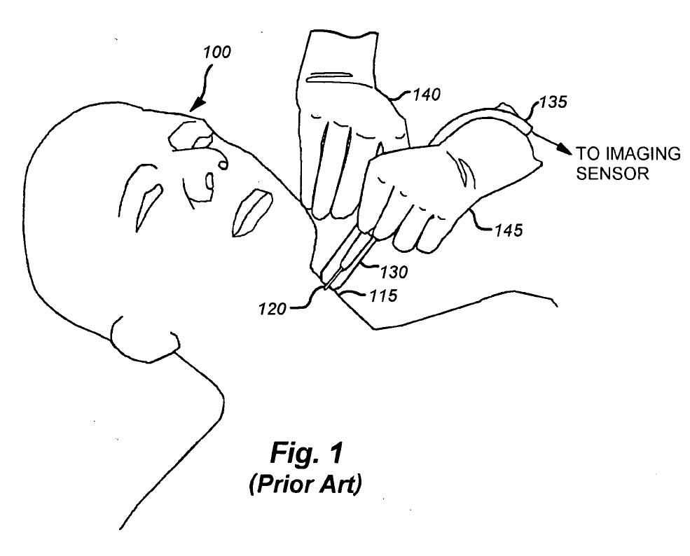

### DETAILED DESCRIPTION OF AN ILLUSTRATIVE EMBODIMENT

### A. Overview and Operation of an Embodiment

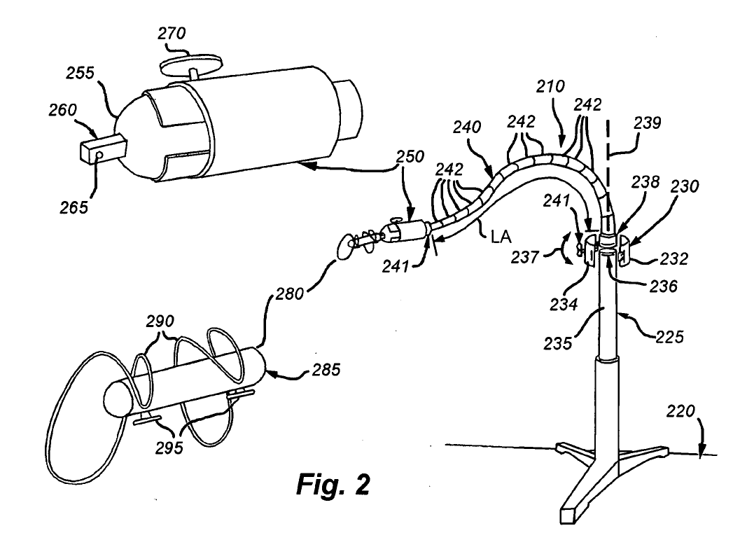

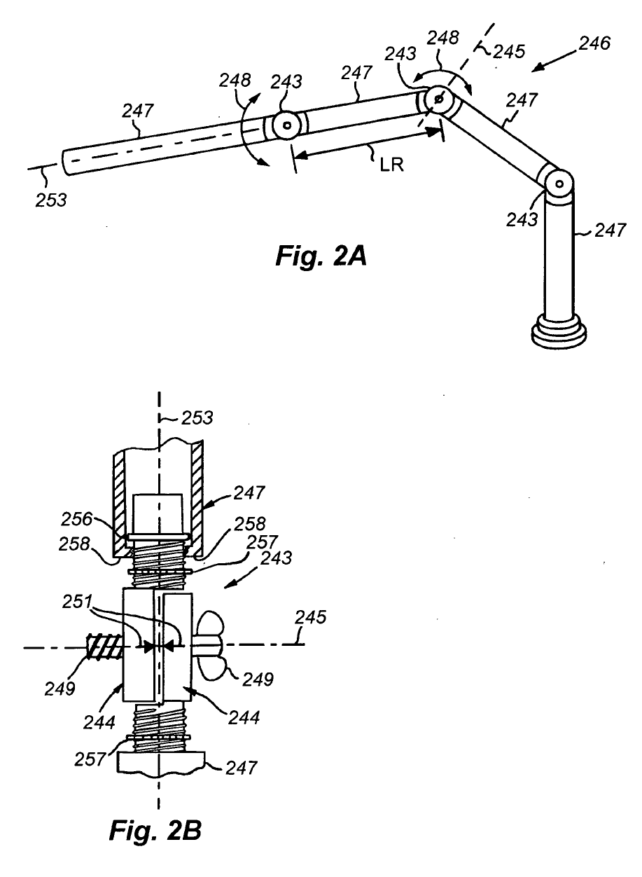

An illustrative embodiment of an apparatus or device for supporting, positioning and stabilizing diagnostic or therapeutic devices is shown in FIG. 2. The overall support device 210 is shown resting atop a rigid, generally flat surface 220 (e.g. a treatment room floor), and is securely mounted to a conventional stand 225, using an illustrative clamping subsystem 230. In this embodiment, the exemplary stand 225 is one normally employed for delivery of intravenous (IV) fluids. However, as described further below, a variety of base units can be used to elevate and suspend the support apparatus of this invention. For example, the support device 210 may alternatively be affixed to other stable or metastable devices or equipment for the duration of the procedure. These may include, but are not limited to, rigid attachment to a hospital bed, backboard, equipment table, or ceiling fixture (see below).

The clamping subsystem 230, in this embodiment consists of a pair of clamp halves 232 and 234 that removably surround and engage the top end 236 of the stand (or other rod-like support member) and the base or proximal end 238 of the support device 210. As described below, additional joints and adjusting mechanisms can be included in the joint between the stand's upright support member 235 and the device base to effect movement and/or adjustment in various degrees of freedom. In this example, the base 238 can rotate (double arrow 237) about the stand axis 239 to effect control of the device's traverse. An associated rotational locking mechanism using, for example a thumbscrew 241, which pressurably engages the base 238 against rotation relative to the stand can be provided.

Operatively connected to clamping subsystem 230 is armature subsystem 240 of the support device 210. In an illustrative embodiment, the straight-line, extended length LA of the armature 240 is in a range of approximately 6 inches to 4 feet. This length is highly variable and is specified, in part, upon the task for which the support device is to be employed and the type and location of the underlying stand/support member to which the support device is attached. As described in detail below, the armature 240 includes a distal end 241 that is adapted to support a medical instrument or device in a wrist subsystem (250 below). It is this medical instrument that a practitioner or operator moves into position against a patient by rotating the base 238 and manipulating the armature to cause it to overlie and engage the region of interest on the patient.

In one embodiment the armature subsystem 240 is constructed from a segmented (see segments 242) flexible metal tubing, commonly referred to as a “gooseneck”. Such a gooseneck subassembly holds a rigid position by frictional forces exerted between each segment 242, which can be overcome by application of pressure by the operator to readjust to relative orientation of the segments. In particular, positioning of the armature by the operator is achieved by grasping the distal end 241 of the armature 240, and forcibly overcoming the holding friction between segments 242 to reorient the gooseneck to a desired position, within the allowed range of movement. This enables the distal end of the armature and its attached instrument to be positioned where needed.

It is recognized, however, that a gooseneck-style metal-segment armature of conventional construction may become more difficult to accurately position at lengths (LA) greater than 1 foot. Additionally, as its length increases, the weight of the gooseneck armature subsystem increases, resulting in a potentially heavy piece of equipment which may collapse if the gooseneck is not able to support its own mass combined with that of the medical device being positioned. Furthermore, when a gooseneck component is manufactured to accommodate greater mass while holding a rigid position, the radius of curvature, and thus position options available to the distal end of the armature subsystem diminishes greatly. Thus, use of gooseneck component is not recommended for armature subsystems greater than two feet in length.

Another illustrative embodiment for armature subsystem 240 is constructed from a tubing assembly consisting of a series of interlocking plastic tube pieces. It has been recognized that such tube interlocking plastic tube pieces are generally light and can be joined together in stable lengths over three feet. Individual pieces lock together tightly, allowing for motion when direct force is applied, but otherwise maintaining the desired position. As the interlocking pieces are hollow, they may provide a pathway for wiring or cabling extending from, and required for, the operation of the medical device being manipulated, preventing said cabling from interfering with the medical procedure at hand. A system and method employing such interlocking plastic pieces is described in further detail below.

According to an alternate embodiment shown generally in FIG. 2A, the armature subsystem can comprise a rigid jointed arrangement 246. This embodiment consists of two to four rods 247, each defining a length LR of between eight inches and two feet, which essentially define a small number of long, jointed segments. The rods 247 are interconnected by joints 243 that allow rotation (curved arrow 248) about at least one bending axis 245. In particular, as shown further in FIG. 2B, the joints 243 comprise a pair of rotating joint halves 244 with an axis pin or screw 249 passing between joint halves 244. This screw 249 can allow the joints 243 to lock by tightening compressing (arrows 251) the halves 244 together. Appropriate knurling or teeth can be provided at the halves' respective confronting faces to generate holding/locking friction between the halves when the screw 249 is tightened. Springs and other structures can be applied to each joint 243 to apply continuous but unlocked friction so that the structure can be relocated, but hold its shape until locked. In some embodiments, friction alone may be sufficient to maintain position without locking. The length of each independent rod 247 can vary with respect to other rods—for example, the rod lengths can be arranged to facilitate closer joints near the distal end. To enable free movement in three dimensions the rods 247 are free to rotate about the axial direction (the direction of elongated extension along axis 253).

As shown further in FIG. 2B, the rod is captured by a flange 256 on the joint end. Then an opposing locking ring 257 is threadingly mounted on the joint end. The ring 257 can be rotated to compress the shoulder 258 at the rod end against the flange 256 to rotationally lock the rod with respect to the axis 253. Again, a friction-bearing structure can apply resistance that can be overcome by sufficient rotational force (e.g. by an operator's hand) when unlocked so that the present orientation of the rods can be maintained after adjustment. In general, each joint 243 is lockable or unlockable independently of the rest of the joints, allowing for complete manipulation. In an alternate embodiment, the joints can be constructed from balls and sockets or similar structures that allow bending along at least two orthogonal axis and free rotation with respect to the axis (253) of elongation.

Yet another alternate embodiment of armature subsystem 240 comprises a flexible hose which is able to accommodate all ranges of motion when in the unlocked configuration, but which can then be locked by the user upon application of an external stimulus, and which comprises a tensioning cable, pneumatic, hydraulic, or other locking mechanism. This flexible hose and all other non-hollow armature subsystems may incorporate a clamping device cabling or wires along the length of the armature (internally, or via an external guide), in order to prevent the wiring from interfering with the operation at hand. While several alternate embodiments of a flexible and positionally stable armature subsystem 240 have been described, many other alternate configurations would also be suitable for use in the present invention and are within the ability of a person of ordinary skill in the art of the invention to derive. For example, any of the various possible combinations of the options previously described may be employed, such as, for example, a rigid joint terminating in a section of flexible hollow tubes.

As discussed briefly above, the distal end 210 of the armature subsystem 240 supports a wrist-type subsystem 250 for attaching a probe or other medical device (refer below) to the armature subsystem 240 and for permitting fine adjustment during the use of the device. Alternatively, the medical device may be directly attached to armature subsystem 240 and be governed by the same positioning and locking methods used for rough adjustments of the armature subsystem. For illustrative purposes, an embodiment of the wrist-like subsystem consists of ball joint mechanism 255, which affords a hemispherical range of motion to male member 260 with spring-loaded friction locking ball 265. Ball joint 255 is able to hold a fixed position by way of a thumbscrew or other locking mechanism 270, yet is easily repositionable when in an unlocked position. The male member 260 is able to connect and lock into finger-type subassembly 280, which has a mechanical connection to the probe or other rigidly held medical device. In this embodiment, the mechanical connection is female socket piece 285 connected to a set of adjustable straps 290, which may hold the medical probe or other device being held and are secured to socket 280 by wrapping around a series of pegs 295 positioned along the length of tube 280.

In an alternate embodiment, the medical device might be rigidly held by a two or more prong clamp, clip, or adjoining plates that are either padded or unpadded Furthermore the female socket member may be directly incorporated into the medical device in question, providing solid mechanical connection without requiring an additional clamping mechanism.

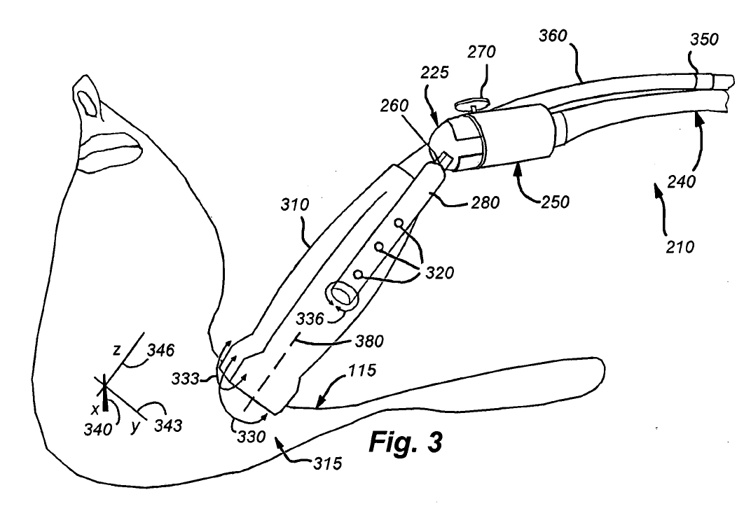

In FIG. 3, a top view of the present invention 210 is shown in relation to a patient's skin surface 115 on the anatomical region of interest 315, and also relative to the previously described armature subsystem 240. Wrist-like subsystem 250 is shown holding an exemplary probe 310, which is being used for an interscalene regional block, as is typically used during surgical procedures for the shoulder or upper arm. Probe 310 is exemplary of the type of instrumentation and process that benefits from hands-free utilization, but it is clear that the present invention may be advantageously employed with any such process or instrumentation known in the art.

The operator initially maneuvers the probe by grasping it (the probe being securely attached to the wrist 250), and overcoming any frictional resistance within the armature 240 and wrist joint 255. The operator, thus, orients the probe 310 at the generally desired position (as defined by the probe's main axis 380 relative to the region of interest 315). Upon alignment of probe 310 with the region of interest, frictional forces within the armature 240 and wrist 250 should be sufficient to enable the operator to release the probe 310 and underlying support device 210. The operator is now able to employ both hands to progress with the underlying medical procedure (e.g. administration of a block, surgery, etc.).

The support device 210 is able to securely hold probe 310 and tolerates fine adjustment or manipulation during the surgical procedure as needed. Fine adjustments are made by applying force to exemplary female socket member 280, which is incorporated directly onto probe 310, either by means of screws 320, by molding the female socket piece 280 with the probe casing, or by other mechanisms of direct attachment. Fine adjustments can be made in any of the rotational directions indicated by arrows 330, 333, 336 by unlocking the position of male component 260 via locking mechanism 270. Rough adjustments made at the armature subsystem level can be made in any of directions 340, 343, 346 indicated by the XYZ-coordinate reference lines. In this exemplary utilization of the device, adjustments may be desirable if a sharper image is required, if imaging of a different anatomical location is required, or if the desired image is lost due to patient movement. Additionally, device 210 prevents loss of sharp image due to operator movement, a primary cause of image loss during unassisted ultrasound guided regional blocks. In this embodiment, armature subsystem 240 has wire or cable clamps 350 connected by means of rivets or other forms of direct attachment at intervals along its length, preventing cabling 360 from hindering the patient or operator during the procedure at hand.

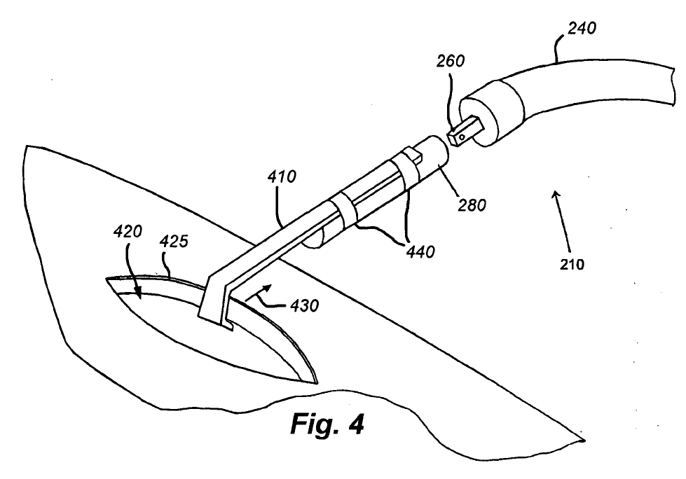

In FIG. 4, another illustrative embodiment of device 210 is shown, in this instance providing rigid support for surgical retractor 410. Retractor 410 serves to expose surgical incision 420 by providing outward force against incision wall 425 in the direction indicated by the vector 430. Upon placement of retraction device 410 within surgical opening 420, present invention 210 is able to hold retractor 410 in place, thereby allowing the operator dual-handed freedom during the operative procedure. In this instance, the medical device is attached directly to female member 280 by a series of adjustable bands 440. Female member 280 locks to male member 260, which is directly connected to armature subsystem 240 and is thus governed by the same positioning and locking scheme of armature subsystem 240. As noted, the locking, positionable armature subsystem may be directly connected to the hospital bed, or to another permanently or temporarily fixed object near the patient, or to the area of focus in the medical procedure.

Having described some generalized features of a support device according to various illustrative embodiments of this invention, structures, features and use-methodologies in connection with particular embodiments of this invention are now described in further detail. Nevertheless, it is expressly contemplated that features and methods described in any portion of the foregoing and following description can be used together according to further illustrative embodiments.

### B. Structure and Function of an Illustrative Embodiment

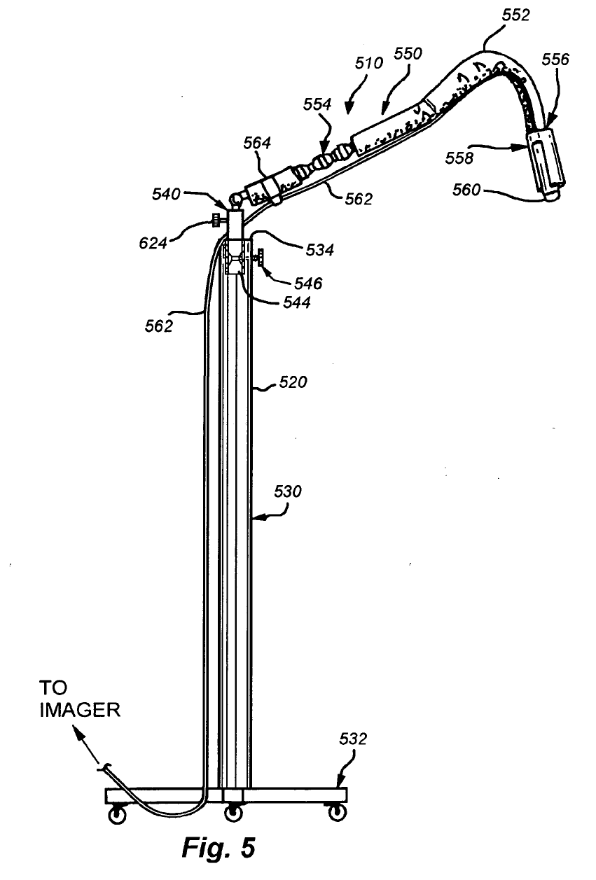

FIG. 5 details an illustrative embodiment of the support device 510 mounted on a freestanding post 520 of a conventional stand 530. The stand 530 can be a type used for suspending lighting, IV drug bags or any other similar supporting operation. The exemplary stand 530 includes a wheeled base 532 that can be locked against movement using conventional wheel locks (not shown). The upper end 534 of the stand 530 includes the mounting base 540, which includes a stem 544 (shown in phantom) that is inserted into a recess on the upper end 534 to secure the support device 510 to the stand 530. The mounting base 540 can be attached to the stand in a variety of ways that depend, in part upon the geometry of the end 534 and other mechanical goals, such as the desired degree to which the device 510 is locked against rotation and separation relative to the end 534. In this example, the stem is secured using a thumb screw 546 of conventional design and function.

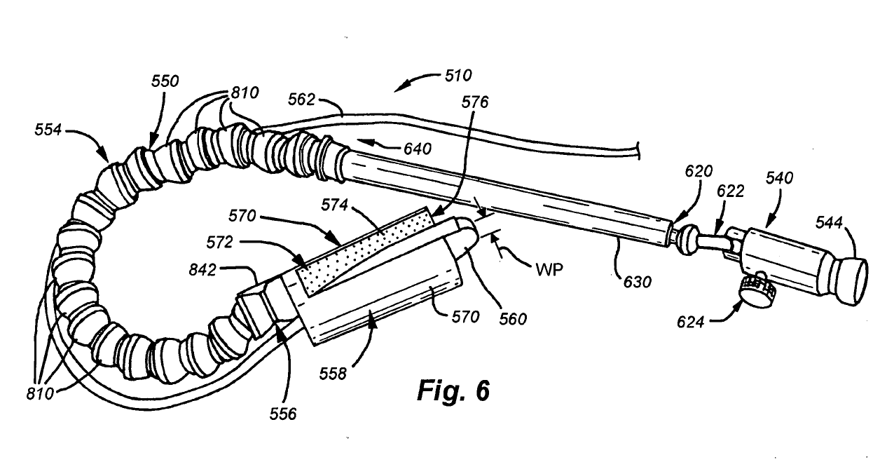

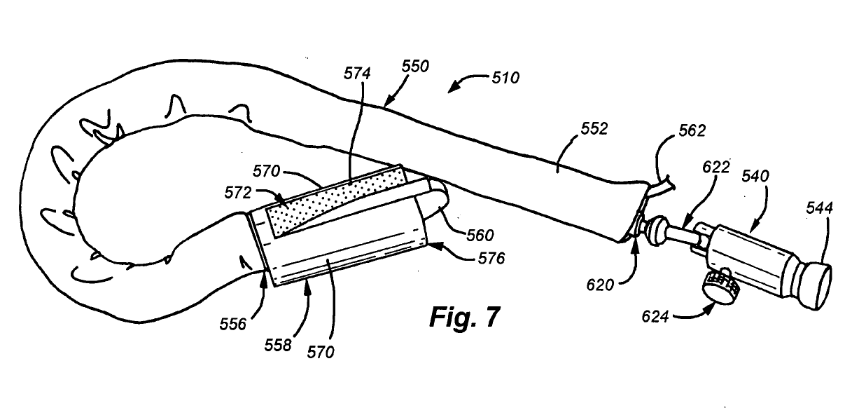

With reference also to FIGS. 6-7, the support device 510 further consists of an elongated, flexible armature 550 that is covered with a sleeve 552. The sleeve 552 has a plastic (vinyl, for example) outer surface that is easy to clean and prevents infiltration of contaminants into the underlying armature segment structure (the sleeve 552 being shown partially exposed in Fig, 5 and removed in FIG. 6) 554. The armature 550, through selective movement the segment structure 554, flexes, rotates and locks in a manner to be described below. The distal end 556 of the armature 550 supports a holder 558 (described further below) that secures a probe 560. In this example, the probe 560 is an ultrasound probe used in connection with the introduction of a needle as described briefly above. The probe is interconnected to an imager of other electronic device via a lead 562. The lead can be run outside the sleeve 552 or tied at various points 564, as shown in FIG. 5. Similarly, the lead 562 can be chased between the sleeve 552 and the segment structure 554 (see FIG. 7). This arrangement prevents possible tangling of the lead 562. Alternatively, as described below, the lead can be chased through the armature interior, where appropriate apertures and/or conduits are provided, and the lead includes sufficiently small-diameter connectors at its imager-end, or at an intermediate location therealong to allow the lead to be passed through the center.

The holder 558 can be adapted to removably secure the probe using a variety of techniques and structures (several of which embodiments are described further below). In one embodiment, shown in FIGS. 6 and 7, the holder defines a U-shaped frame with a pair of halves 570 and an open space 572 therebetween. The space is sized and arranged to allow passage of the probe thereinto. The holder of this, or other embodiments can be constructed from a metal (such as aluminum) or from a durable, low-density polymer, such as nylon, Delrin®, glass-filled composite, etc. A piece of foam or another resilient, frictional material (neoprene, for example) 574 is applied to interior of each half 570, thereby defining an interior space that is smaller than the width WP of the probe. The foam can comprise a “memory” type elastomer that retains the shape applied to it over a period of time to allow for easier insertion and reinsertion of the probe and for accommodation of a variety of probe shapes. When the probe is inserted through the open distal end 576 of the holder 558, it causes the resilient material 574 to elastically deform and pressurably engage the probe 574. The material's engagement is sufficiently frictional to prevent the probe from moving under normal use. The holder is secured to the distal end 556 of the armature 550 using a variety of techniques described further below.

With reference to FIG. 6, the proximal end 620 of the armature 550 is attached to the base via a ball-joint assembly 622. The ball joint assembly is particularly desirable where the patient's region of interest/treatment is located vertically above the level of the joint so that the entire support device can be pivotally elevated above the region. The ball joint assembly also advantageously affords a range of angular movement to the armature to maintain desired pressure on the subject region of interest. The structure and function of the ball joint assembly 622 is also described in greater detail below. In general, its various components can be constructed from one or more durable, long-life materials, such as stainless steel alloy. The ball joint is optionally lockable using a base-mounted thumbscrew assembly 624 in this embodiment (described further below). The structure and function of the ball-joint's locking mechanism (if any) is highly variable according to alternate embodiments.

The ball joint assembly 622 in this embodiment interconnects to a straight lead tube portion 630 of the armature 550. The lead tube portion 630 defines a length in a range of approximately 6 inches to 1 foot in this example. The length of this lead tube portion 630 is highly variable. In alternate embodiments, the straight lead tube is omitted entirely, and the proximal end 640 of the segmented structure 554 of the armature 550 is sized and arranged to mount directly to the ball joint assembly 622. The lead tube portion 630 in this embodiment is adapted to provide a non-flexible base that extends the flexible segmented distal portion of the armature to a location where its flexibility is most useful (i.e. overlying the patient/region of interest).

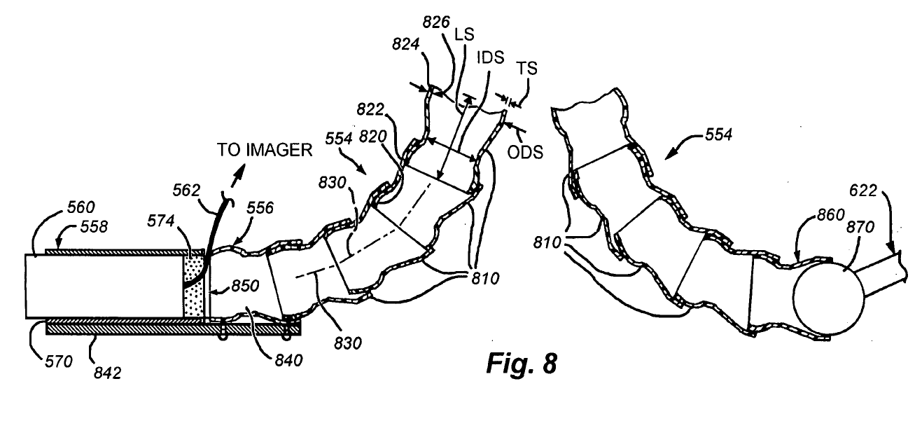

With reference also to the cross section of FIG. 8, the construction of the segmented structure 554 of the armature 550 is shown in greater detail. This structure consists of a chain of interengaging plastic tubular segments 810. Each segment 810 defines a semi-spherical nose 820, a narrowed, cylindrical center section 822 and a flared tail section 824. The inner surface 826 of the tail section 824 defines a semispherical socket that is particularly adapted to slidably engage the semispherical outer surface of the nose 820. The nose 820 and inner surface are sized an arranged to allow each engaging segment's tail 824 to bend in any radial direction about the adjoining segment's nose 820 to a maximum angle of approximately 10-15 degrees in an illustrative embodiment. Similarly each segment can freely rotate with respect to the other about their common axis 830 (taken through the center of each segment). When connected in chains, the segments allow a multi-directional bend of virtually any angle or combination of angles in any direction in three dimensions. This allows the holder 558 to be oriented against the region of interest at a precise desired angle-of-attack. The ability to rotate the segments relative to each other affords a further rotational degree of freedom that can be exploited at the distal end to orient the holder 558 at the precise desired rotational angle.

In this example, each segment is constructed from a durable, elastically deformable polymer, such as polyethylene, ABS, polyester or polyvinylchloride (PVC). The mating of adjoining segments 810 is essentially fluid tight. In an exemplary embodiment, each segment defines an overall length LS of approximately 1-1½ inches, a maximum outer diameter DS of approximately 1-1¼ inches and an inner diameter IDS of approximately ¾ inch. The wall thickness TS varies between approximately 1/16 and ⅛ inch. These dimensions are highly variable in alternate embodiments. A commercial version of unmodified segments is available under the trade name Loc-Lines® from Lockwood Products, Inc. of Lake Oswego, Oreg. (¾″ modular tubing system).

The number of segments 810 used to construct the armature 550 is highly variable. In exemplary embodiments, approximately 15-25 segments can afford desired flexibility over a range of 1½-2½ feet. The precise number of segments is highly variable and depends upon the desired mounting arrangement and accessibility of the region of interest.

As described above, the holder 558 is attached to the distal end 556 of the segmented section 554. The most-distal segment 840 includes an attached bar 842 that is also secured to the shell of the holder 558. This is only one of a wide variety of attachment mechanisms that should be clear to those of ordinary skill. In alternate embodiments, the proximal end 850 of the holder 558 can be joined directly to the distal segment 840 using, for example a threaded coupler. Axial rotation of the holder can be provided by the general capability of segments 810 to rotate with respect to each other along the chain, or by one or more specialized wrist joints at the holder-to-distal segment junction and/or at other locations along the armature. Such wrist joints can incorporate a frictional brake or other rotational lock.

Similarly, the most-proximal segment 860 is joined directly to the ball end 870 of the ball assembly 622 in this example. This arrangement omits the lead tube portion 630 described above. Conversely, the ball assembly can be fixedly attached to the proximal segment or an included lead tube 630. The entire arrangement between the ball joint assembly and the holder can define a continuous lumen through which tubing, electrical leads and other elongated structures can pass (refer below).

The segments 810, when assembled, allow reorientation of the holder 558 and probe 560 with a modicum of applied force. Once reoriented, the interengaging segments typically exhibit sufficient frictional holding force therebetween to maintain their position. However, for many applications, particularly where longer-length armatures are employed (approximately 1½ feet or greater) there may be some risk of dislocation of the holder due to weight-induced movement/sagging of the armature. To ensure that the armature remains securely oriented against the region of interest after adjustment, it is often desirable to ‘lock’ the position of the armature by providing additional frictional force (or other holding force) between adjacent segments.

C. Armature Locking Mechanisms

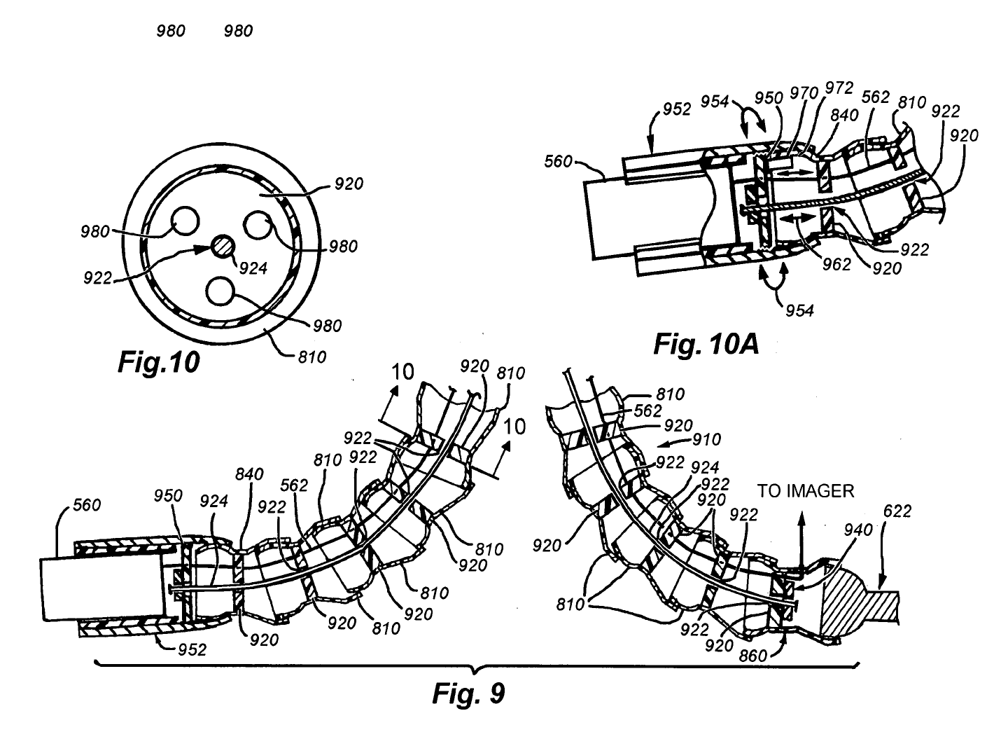

FIGS. 9, 10 and 10A detail an illustrative embodiment of an armature locking assembly that causes the segments 810 to retain their current shape against increased force thereon. When unlocked, this predetermined level of force would otherwise cause the segments to bend relative to each other. The segmented structure 910 includes a plurality of segments 810 that are similar to or identical to those described above. Each segment in this embodiment is provided with an internal disk or “bulkhead” 920 that is fixedly secured to the inner wall of the segment 810 at an appropriate location. The disks 920 can be secured using friction, adhesives, welding or fasteners. Each disk 920 includes a central passage 922 centered on the segment's central axis (830 in FIG. 8). The central passage 922 is sized to allow a cable 924 pass therethrough with minimal radial play. The cable 924 can comprise any acceptable metal, composite or polymeric material according is to an illustrative embodiment. The cable is typically inelastic and can be composed of a monofilament or stranded metal. The cable 924 is axially anchored at the proximal end by an anchor disk 940. A base disk 950 is axially fixed to the distal end of the cable 924. The base disk includes external threads that engage mating internal threads of the holder 952. As such, the distal end of the cable 924 can be moved axially by rotation of the holder 952 which threadingly engages a threaded base disk 950. In this manner, rotation (curved arrows 954) of the holder 952 causes the threaded disk to move axially (double arrow 962) relative to the distal end segment 840 of the segmented structure 910. The base disk 950 can be rotationally fixed to the distal segment 840 by one or more blades 970 (FIG. 10A) that engage corresponding slots 972 in the distal segment 840.

Thus rotation (curved arrows 954) of the holder by the user can apply and/or release tension in the cable 924. When tension is applied, the cable 924 exerts a compression force over the entire structure. The disks 922 are located so that the tension is resolved into axially directed compression force between segments. Hence the entire segmented structure 910 is placed under increased compression at respective joints between segments. This compression increases the applicable friction force and serves to lock the entire structure in its current position against movement under moderate force (e.g. force equal to or somewhat greater than the predetermined force needed to reorient the armature when unlocked).

In operation, the practitioner moves the holder to the desired orientation with respect to the region of interest, then rotates the holder until it can no longer move without rotating the underlying segments. Further rotation of the holder causes the segments to move as well. The force applied by the now-tensioned cable should be sufficient to maintain the structure in a desired orientation. Note that the disks are provided with one or more apertures 980 through which one or more leads 562 can be passed. The disks can be rotated relative to each other a moderate degree without binding a lead passing therethrough. In alternate embodiments, a flexible conduit carries leads through the disks, or leads are carried outside the lumen of the armature as described above (see FIG. 5). In another embodiment, the disks 920 can be mounted on rotational bearings (not shown) that fix the disks in an axial direction relative to respective segments, but that allow free rotation so that any chased leads passing through the apertures 980 do not bind between is segments as the segments are rotated axially with respect to each other.

Note that the proximal end of the segmented structure 910 in the embodiment of FIG. 9 includes a fixed (non-rotating/bending) attachment to the ball joint assembly 622.

While a holder-mounted tensioning system is shown and described in this embodiment, it is expressly contemplated that the tensioner can be a separate member mounted on the holder or at another location (such as the proximal end) on the armature. The tensioner can be manual, or can be automatic, powered by a motor with appropriate limit stops and/or tension sensors to regulate motion. Where automatic, the switch can be mounted on the holder (see below) or at another convenient location (for example, a foot pedal).

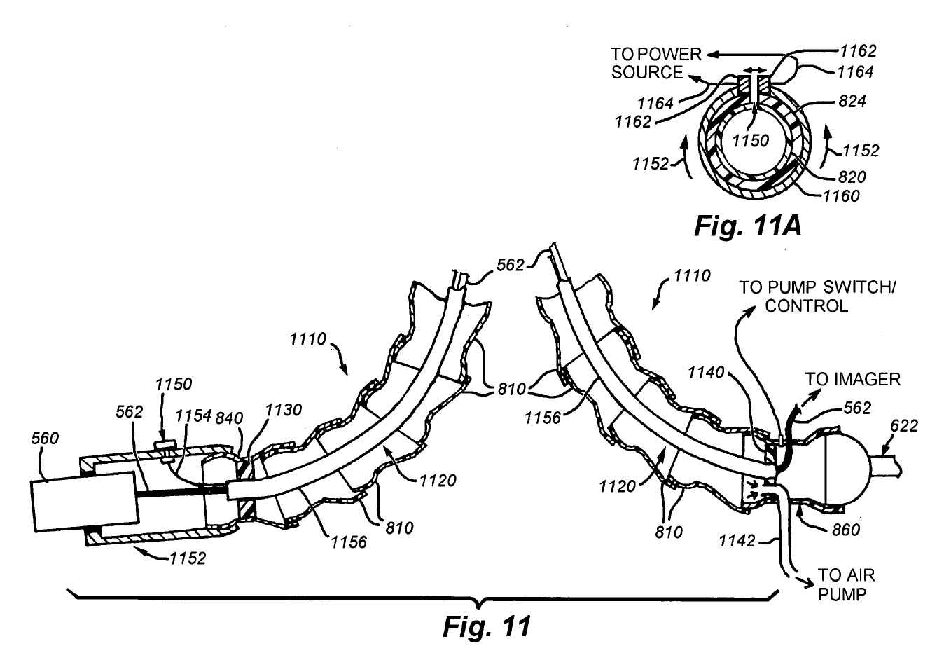

According to another embodiment, the locking mechanism can be based upon the relatively fluid-tight interconnection between segments 810. An illustrative embodiment of the armature locking mechanism based upon evacuation of air (a vacuum) is shown and described in connection with FIG. 11. The segments 810 are interconnected into a segmented structure 1110 that defines an inner volume 1120 that can be evacuated to create a negative pressure balance with respect to outside, ambient air pressure. The volume is evacuated by sealing the distal segment 840 and proximal segment 860 with respective sealing disks 1130 and 1140. The proximal sealing disk 1140 communicates with a vacuum pump (not shown) that receives control signals from a switch 1150 mounted on the holder 1152. Pressing the switch either directs release of the vacuum or application of the vacuum. A lead 1154 runs through the inner lumen of the volume 1120 via a sealed conduit 1156. The conduit 1156 isolates the evacuated volume 1120 from the ambient air, being sealed at the distal and proximal sealing disks 1130 and 1140, respectively. The conduit 1156 also carries the electrical lead 562 for the probe 560. The conduit is sufficient flexible in torsion and/or appropriate sealed bearings are provided to the disk or conduit to allow segments to rotate. Conversely, rotation is allowed where the holder engages the distal segment 840 to provide sufficient rotational flexibility for the support device of this invention.

In operation, where the vacuum is engaged until the switch is activated, the practitioner presses the switch whenever he or she wishes to reorient the armature. The vacuum is then released and movement is permitted. When the switch is released, the vacuum is quickly restored and the segments bear upon each other with nearly 14.7 psi, significantly increasing the inter-segment frictional force. This serves to fix the segments with respect to each other. Conversely, where the vacuum only activates after the switch is pressed, the practitioner refrains from pressing the switch while reorienting the holder position to a desired orientation. The switch is then depressed to activate the vacuum and lock the arrangement at the chosen orientation. In each example, the frictional holding force can be enhanced by providing a roughened surface to at least one of the interengaging surfaces of the segment joint. However, the roughening should allow enough sealing to maintain the desired vacuum level.

In any of the locking embodiments described herein actuation of locking/unlocking can be accomplished using a switch or knob on the holder, body of the armature or stand or via a remote switch such as a foot pedal. Alternatively, using existing voice-recognition and computer interface technology, it is contemplated that locking/unlocking can occur via voice-command with appropriate locking/switching actuators interconnected to the voice-recognizing computer interface.

Another locking mechanism is detailed in cross section in FIG. 11A, taken through a joint between interengaging segments 810. This arrangement employs hoop stress on the tail section 824 of one segment against the nose 820 of a mating segment. Appropriate friction-enhancing surface finishes (roughening, crenellations, etc.) can be provided to one or both mating surfaces. The tail section 824 is provided with an axially directed split 1150 so that it can move circumferentially (curved arrows 1152) to constrict around the inner nose section 820. In this embodiment, the split tail section is provided with a stress ring 1160 constructed from metal, composite, or another relatively sturdy material. The stress ring is also split and includes a pair of actuator halves 1162 at the split that are controlled by leads 1164. The actuator halves 1162 can be electromagnetic, hydraulic, pneumatic, or mechanical. The leads 1164 transmit the appropriate power from a switched source (under operation of a switch (see switch 1150 above)) to allow the actuator halves to move toward and away from each other. As they move toward each other they increase hoop stress and frictionally lock the tail section 824 to the nose 820. Releasing the actuators releases the hoop stress, and thereby unlocks the joined segments. This arrangement is applied to every segment junction that is lockable within the overall armature. This arrangement has the advantage of allowing selected segments to be locked at certain times within the orientation procedure. For example in an exemplary orientation procedure: the more-proximal segments are locked first and more-distal segments are locked subsequently, allowing for final tuning of the holder's orientation after gross positioning of the armature has occurred.

It should be clear that other locking mechanisms that apply a holding force between selected joints between segments are expressly contemplated. For example, a system of moving pistons or bars can be deployed on each tail section to bear upon an interengaging nose section, thereby exerting a locking force.

D. Ball Joint Assembly

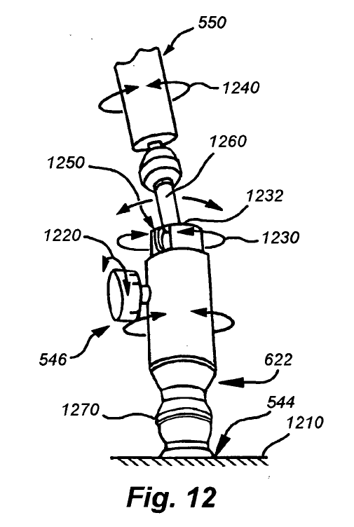

The base of the armature consists of a ball joint assembly. FIGS. 12-14 further detail the construction of the ball joint assembly 622 according to an illustrative embodiment. The stem 544 of the ball joint assembly 622 is keyed or otherwise secured against pull-out and rotation into the chosen base structure 1210 as shown. The outwardly exposed portion of the ball joint assembly 622 includes a locking thumbscrew assembly 546. The thumbscrew is rotatable (double curved arrow 1220) to selectively lock and unlock rotation (double curved arrow 1230) of the upper ball mounting 1232 of the assembly 622. This rotation essentially regulates the swing of the entire armature about the base structure 1210 (e.g. a stand, wall, ceiling, imager housing, etc.). The additional jointed segment 1270 located proximal to the ball joint mechanism 622 may be included to serve as a strain relief or to further extend the range of motion achieved by the distal end of the device.

As detailed in FIG. 14, a pair of internal wedges 1410 (housed within the lower assembly housing 1411) are directed inwardly (arrows 1412) upon rotating the thumb screw 546 to lock the rotation. The wedges 1410 cause the internal frustoconical base 1420 of the upper ball mounting 1232 to move upwardly (arrow 1422) so that its inner shoulder 1424 comes into engagement with the shoulder 1430 of the lower assembly housing 1411. This engagement frictionally locks the lower assembly housing 1411 to the upper ball mounting 1232, thereby preventing relative rotation therebetween. The lower assembly housing 1411 is, likewise, locked against rotation and pullout with respect to its base, stand, etc. Thus, the support device is effectively locked against rotation. Note that the thumbscrew assembly 546 is only one of a variety of possible locking arrangements.

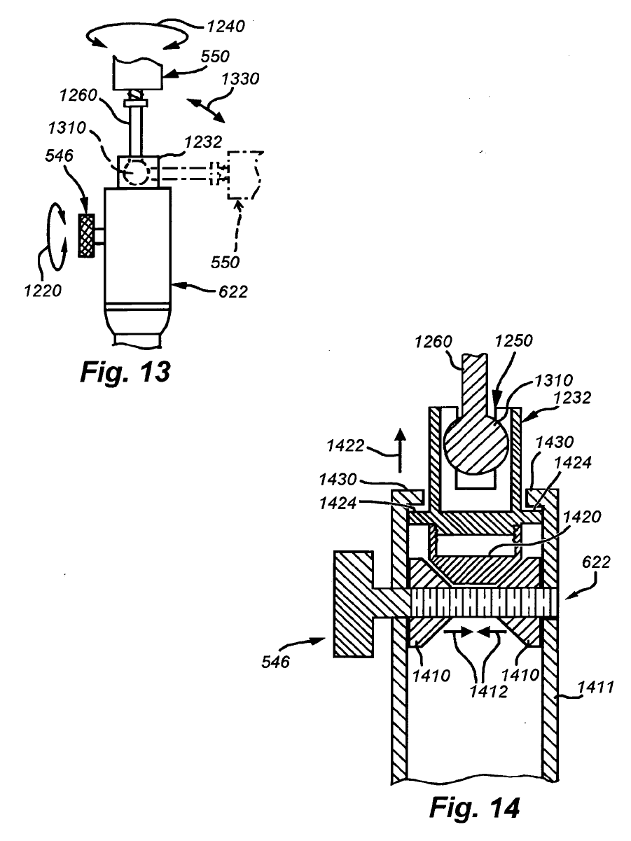

The upper ball mounting 1232 supports a free-floating ball 1310 (FIG. 13) that engages the proximal end of the armature 550. In this embodiment, the ball allows the armature to axially rotate (curved arrow 1240 in FIG. 12) relative-to the housing. This feature can be omitted in favor of a non-rotating hinge assembly in alternate embodiments. Significantly, as shown in FIG. 13, the upper ball mounting includes a slot 1250 sized to allow the distal stem 1260 of the assembly to pivot (double arrow 1330) between a vertical and horizontal position (shown in phantom in FIG. 13). In this embodiment, the pivotal motion is unlocked. As described below, this allows the assembly to hinge downwardly under its own weight to bear pressurably against the region of interest, with all other degrees of freedom being locked against inadvertent movement.

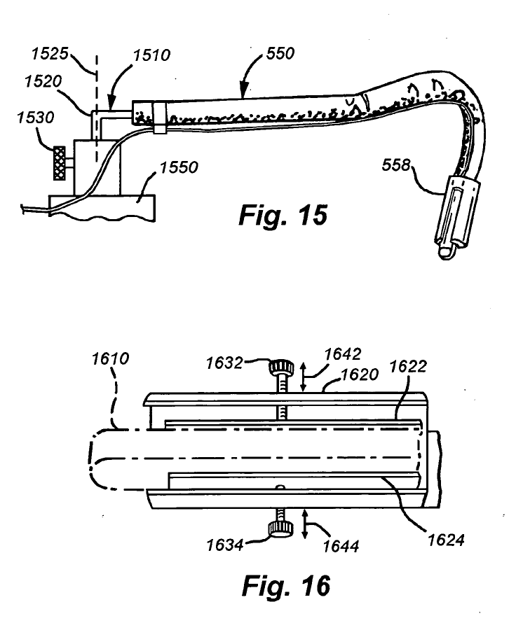

Alternatively, FIG. 15 details a flexible armature 550 and probe holder 558 as described above with a non-hinged base assembly 1510. In this example a fixed L-shaped (or other appropriately shaped) elbow/base member 1520 extends from a rotational lock (thumbscrew) assembly 1530. This lock can be similar to that shown in FIG. 14. The lock is mounted on a stand or other base structure 1550. This arrangement is capable of rotation about the depicted base vertical axis 1525. In this embodiment a floating ball joint assembly is omitted (a floating hinge can be included in alternate embodiments) and the holder relies upon positive pressure applied by the operator to flex the armature and thereby maintain contact with the patient. The pressure is maintained through the frictional engagement of armature segments or by activating a locking force in the armature. Such an L-shaped joint may be advantageous when the base extension of the support device (the extension tube) projects outwardly above a recumbent patient. A low-friction bearing can be applied to this or other embodiments herein to facilitate rotation about the base vertical axis 1525.

E. Instrument/Probe Holders

The exemplary probe holder described above employs elastically deforming, frictional material to firmly grip the probe at a desired angle. A variety of mechanisms for engaging and holding the probe are contemplated. As shown in FIG. 16 a probe or other instrument 1610 (shown in phantom) is mounted within a holder shell 1620. The probe is secured between two pressure plates 1622 and 1624 that are each brought into and out of pressurable contact with the probe/instrument 1610 by rotating respective thumbscrews 1632 and 1634. Each thumbscrew 1632, 1634 is rotated to move (respective arrows 1642, 1644) the attached pressure plate 1622, 1624 relative to the fixed wall of the shell. The inner surface of each pressure plate can include a frictional surface (such as rubber or neoprene) to provide increased holding force to the probe/instrument.

A variety of alternate mounting arrangements and actuators for applying hold force (e.g. springs, levers, etc.) are expressly contemplated according to alternate embodiments. For example, in an alternate embodiment an inflatable gel or fluid (air, for example) bladder can be employed to selectively retain the probe in the holder. In general the holder should be sized so as to comfortably fit in the practitioner's hand. In this embodiment, a holder that is between approximately 3-5 inches in length and 1½-3 inches in diameter (or approximate maximum width in the case of a non-circular cross section).

F. Support Device Base Structures

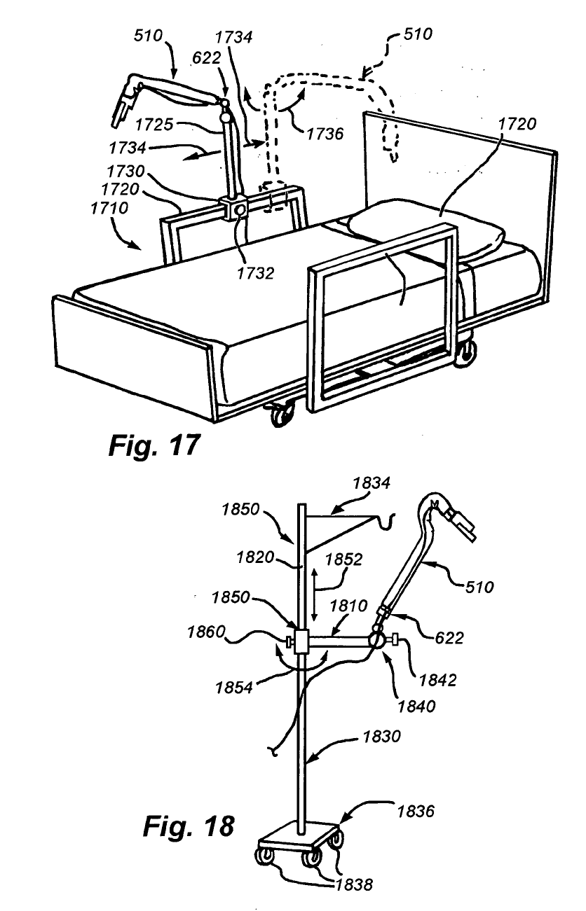

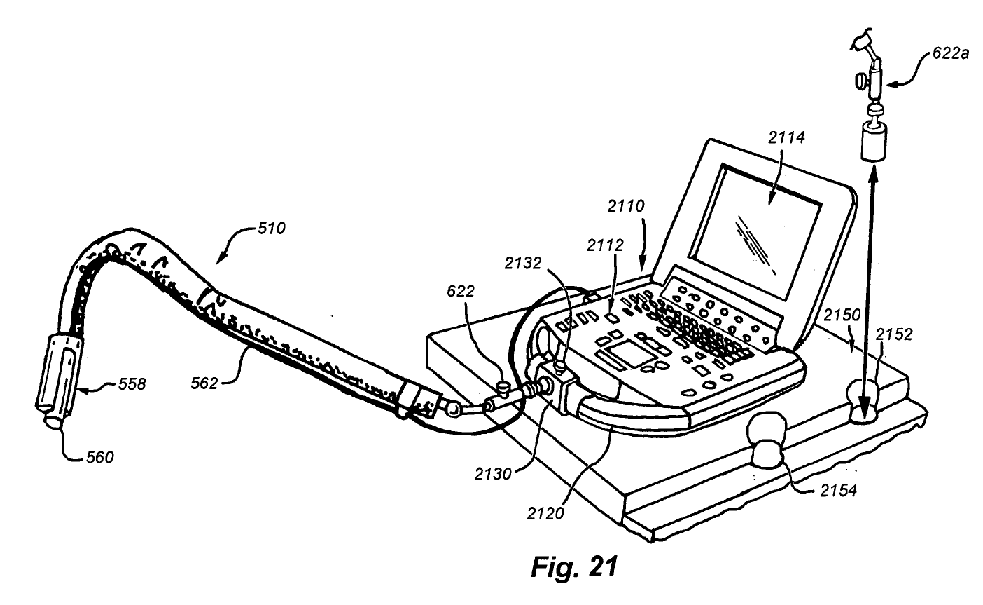

As described above, the support device according to the various embodiments herein can be mounted to a variety of fixed or movable structures to effect appropriate positioning with respect to the patient's region of interest/treatment. FIGS. 17-21 detail various exemplary mounting/basing arrangements for the exemplary support device 510 (or any other device embodiment described herein) according to this invention. As described above, the overall length of the device 510 can be varied (by adding or deleting segments and varying or omitting extension tubes) to generate the desired overall length and geometry needed for a particular mounting location and attitude.

FIG. 17 shows the placement of the support device 510 with respect to a typical hospital bed 1710 with side railings 1720. The device 510 is mounted at the top of a vertical extension tube 1725 placed on a clamping base 1730 that can define a C-shaped clamp. An appropriate locking mechanism, actuated by a thumbscrew 1732, for example, is used to lock the base 1730 with respect to one of the side rails 1720. The mechanism applies predetermined friction against the rail, or otherwise secures the base against detachment and lateral sliding (arrows 1734). The base 1730 can be relocated as shown in phantom by releasing clamping pressure and sliding it to another exemplary position (shown in phantom). Likewise the base permits swiveling of the support device (curved double arrow 1736) to overlie the bed area. The swiveling motion can be locked using a thumbscrew or other rotational mechanism as described above.

FIG. 18 shows another, more-portable implementation of a device-basing arrangement. In this example, the device 510 is attached to a base arm 1810 that extends transversely (perpendicularly in this example) relative to a freestanding post 1820 of an 20 IV stand 1830. The stand 1830 is shown containing a top hook-,assembly 1834 for hanging IV bags and a wheeled base 1836 that may include lockable caster wheels 1838 or another type of floor brake to restrict motion once the stand 1830 and attached support device 510 is positioned appropriately with respect to the patient/region of interest. The transverse base arm 1810 supports the device ball joint assembly in a gudgeon or other well-shaped aperture that engages the assembly's distal mounting stem (see stem 544 in FIG. 5) and that is lockable against pullout and rotation using, for example, a thumbscrew 1842. The post-end 1850 of the base arm 1810 engages the stand post 1820 in a manner that allows it to be adjusted vertically therealong (double arrow 1852) and rotated about the post's axis of elongation (curved double arrow 1854).

The base arm post-end 1850 is shaped to at least partially surround the arm. It can be a full or partial cylinder. Where it is a partial cylinder, it can include a slot (not shown) that allows it to pass over the hook assembly 1834 upon attachment to the post. It can be locked using a thumbscrew 1860 or similar locking mechanisms. Alternatively the post-end can comprise at least two half-shells that can be selectively compressed against the post 1820 in a desired position to hold the base end in place thereon. When released, the half shells can allow the device to be reoriented vertically and rotationally on the post 1820, or attached to and detached from the post 1820. Additional adjustable degrees of freedom can also be provided to the base arm 1810 along with provision of appropriate locking mechanisms for such additional degrees of freedom.

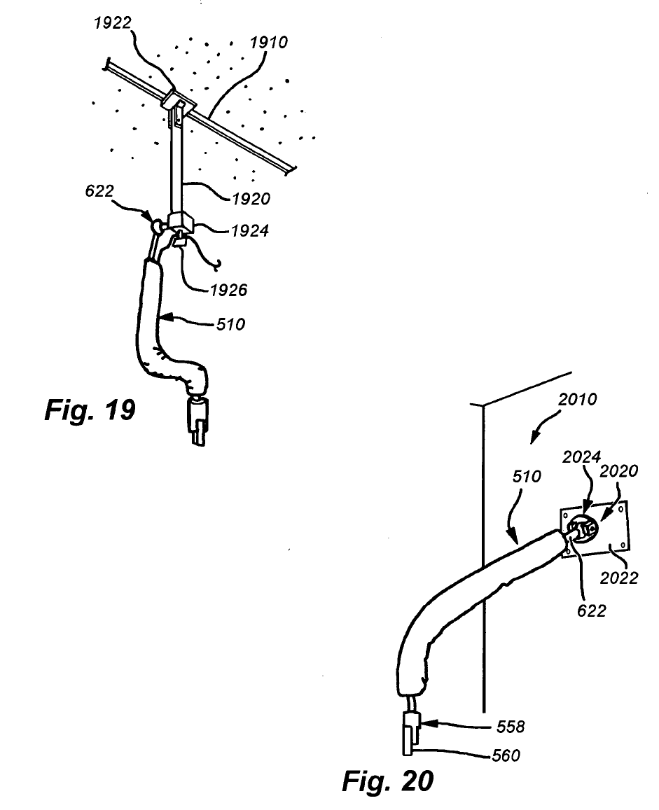

It is also contemplated that the support device 510 can be attached to a variety of fixed surfaces including walls, cabinets, ceilings, etc. As shown in FIG. 19, the device 510 is mounted from a ceiling track 1910 that can be a purpose-built equipment rail or a sufficiently sturdy drop-ceiling support. A vertical extension post 1920 extends downwardly from a proximal track clamp 1922. This track clamp 1922 is adapted to removably and adjustably lock to the track 1910 at a desired location. The distal end of the extension post 1920 includes a mounting base 1924 and thumbscrew 1926 that receives and locks the ball joint assembly (or another armature base structure) in a manner described generally above. The mounting base can be adapted to rotate and lock with respect to the axis of extension of the post 1820 and/or the ball joint assembly 622 can be mounted on-axis with the post (from the bottom of the base 1924) in alternate embodiments, rather than from a side as shown.

FIG. 20 details another implementation of a fixed-base mounting arrangement in which the support device is mechanically connected to a vertical or upright fixed surface, such as a wall 2010. The mounting base 2020 in this example consists of a wall plate 2022 that is bolted, or otherwise attached to the wall surface 2010 at an appropriate height and placement relative to a treatment area (a gurney, bed, examination table, etc.). The mounting base 2024 includes a base structure 2020 that engages the ball joint assembly 622. The assembly 622 can be mounted in a horizontal or vertical orientation (or an angle therebetween) depending upon the geometry of the receiving aperture of the base 2024. The base can include a lock, and allow rotation in one or more degrees of freedom. In alternate embodiments, the ball joint assembly can be mounted on a sliding structure (such as a tube or rod) that allows vertical and/or horizontal adjustment of the location of the ball joint assembly relative to a wall. In general, the depicted wall-mounting arrangement and other arrangements described herein are provided with a floating ball joint in an illustrative embodiment, that permits the holder 558 and probe 560 to rest on the region of interest under predetermined weight-generated pressure. Hence the overall assembly is locked in a predetermined orientation via the mounting base, and the ball joint remains free to float within a given range of angular/pivotal motion to generate a resting pressure in the general direction of gravity.

The support device according to various embodiments contemplated herein can be integrated with an imaging or other medical-electronic system that can benefit by the addition of an instrument-holding device. As shown, the support device 510 (or other device embodiments described above) with holder 558 and probe 560 is mounted with respect to a commercially available ultrasound scanning and imaging system 2110 including a user interface 2112 and display 2114. In this example, the display is used by the practitioner to image the region of interest engaged by the probe 560. This imaging system 2110 includes an integral carry handle 2120. A clamp or permanent mounting base 2130 is attached to the handle 2120 using a locking thumbscrew (in detachable versions of the base) 2132. The ball joint assembly 622 extends from the base 2130 horizontally in this example. It can extend vertically or at other angles in alternate embodiments. While the base 2130 is mounted on a handle 2120 in this embodiment, it is expressly contemplated that it can be mounted at other convenient locations on the equipment piece. Furthermore, where the underlying equipment is placed on a support cabinet or stand 2150, the stand, itself, can include one or more mounting points 2152 for receiving the ball joint assembly (622a). The underlying equipment, likewise, may be part of a large housing that is not portable by hand-carriage (it may be wheeled, however).

In this embodiment, the lead 562 connecting the probe 560 and the imaging system 2110 is carried externally of the support armature. As described above, the lead can be carried internally. In fact, the lead can be integrated into the structure of the armature using embedded conductors and other mechanisms in alternate embodiments. Similarly, it is expressly contemplated that the support device base can include an integral connector system so that the probe is automatically connected to the imaging system when the device is mechanically secured to the imaging system. Appropriate plug connectors, fiber optic links and/or other interengaging structures can be provided between the support device base (ball joint assembly) and the mounting base to make the connection.

While each of the foregoing embodiments references an interconnection between a mounting base and the ball joint assembly, it is expressly contemplated that a non-floating, proximal support device base can be used in connection with the mounting base in alternate embodiments. Hence, the term ball joint assembly as used in connection with the description of various mounting arrangements should be taken broadly to include non-hinged/floating structures, unless otherwise noted.

G. An Exemplary Procedure Employing the Support Device

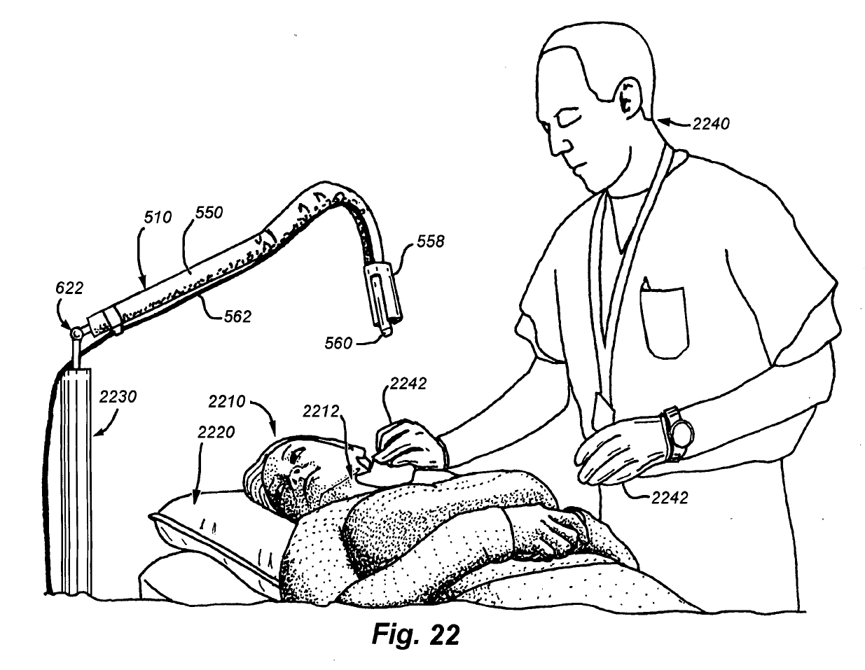

FIGS. 22-33 detail various steps of an exemplary procedure in which the support device of an embodiment of the invention is employed to assist the guiding of an anesthetic neural-block-delivery needle into a patient's region of interest/treatment. This procedure contemplates further surgery in the area of treatment following the application of the block. In alternate embodiments, the depicted procedure can be the primary treatment administered to the patient.

As shown in FIG. 22, the procedure begins with the patient 2210 reposed on a stable surface (bed 2220). The patient 2210 is sufficiently immobilized using sedatives and, where appropriate supports or other restraining devices so that the region of interest/treatment (in this example, the neck region 2212) will not move appreciably during the procedure. A local anesthetic may also be applied to the region 2212. The practitioner 2240 has applied sterile gloves 2242 to his or her hands and avoids any non-sterile surface, or bringing any non-sterile surface into contact with the patient 2210. Subsequently the practitioner 2240 applies a sterilizing wipe 2244 to the region of interest/treatment 2212. The support device 510 (or any other embodiment of the support device contemplated herein) is positioned adjacent to the region 2212 of the patient 2210. In this example the device is mounted via the ball joint assembly 622 on a freestanding post 2230 of a floor stand. The device is oriented so that the armature 550 is suspended over, and out of contact with the patient 2210 as the practitioner 2240 prepares the region 2212 for the procedure. As such the holder 558 and probe 560 are located at a distance above the patient's region of interest/treatment 2212.

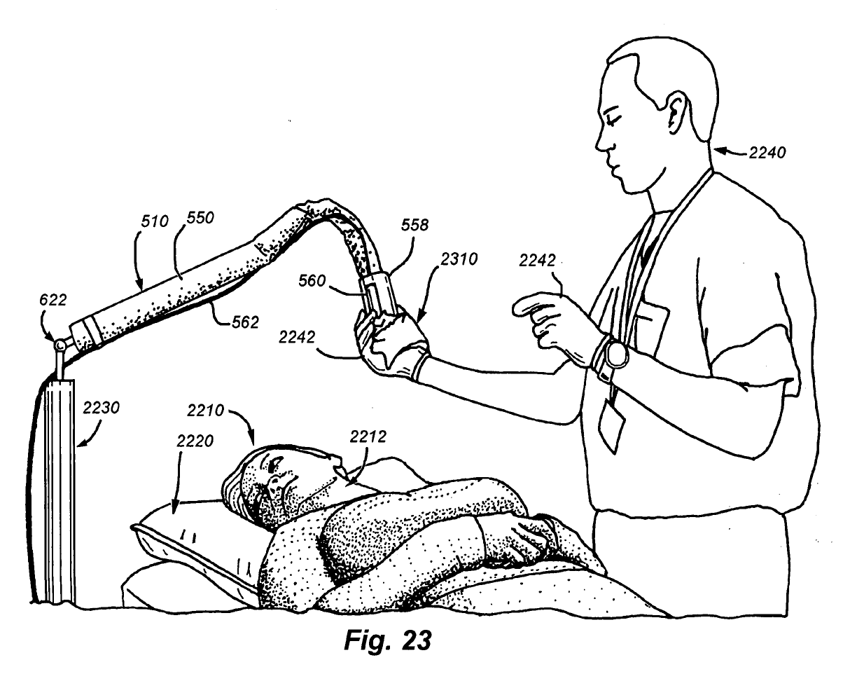

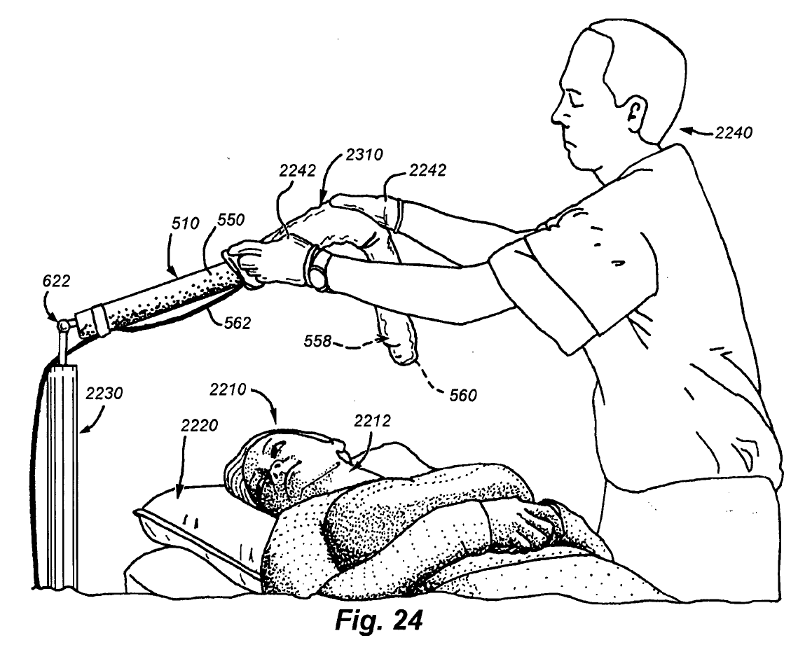

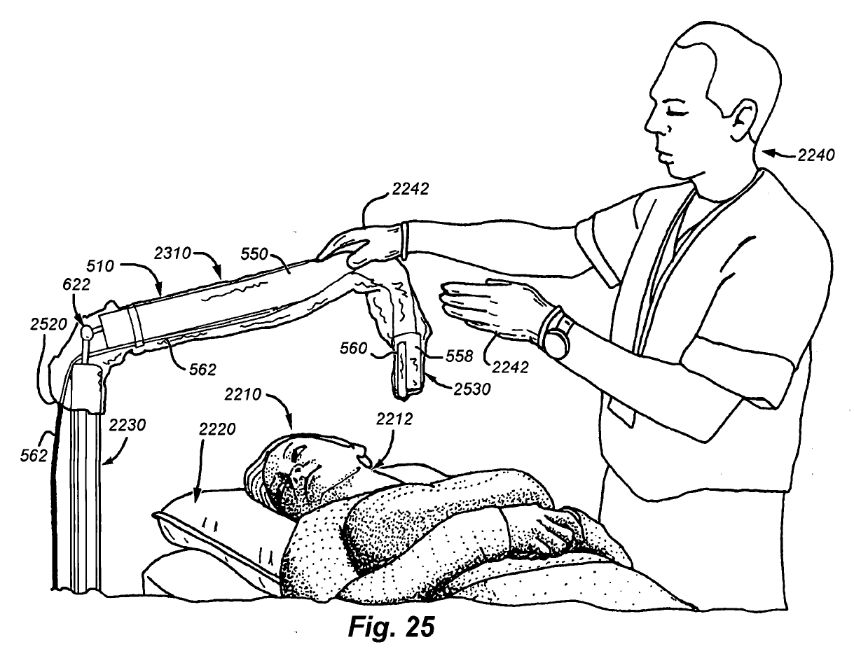

The next step in the procedure is detailed in FIG. 23. Since the probe 560 and holder 558 should remain sterile when contacting the patient 2210, the practitioner 2240 now drapes the probe 560, holder 558 and armature 550 in a continuous elongated drape 2310 with an open proximal end and sealed distal end. The drape is constructed from a transparent biocompatible, sterile polymer, such as polyethylene sheet. As shown further in FIG. 24, the drape 2310 is shaped and sized to fit over the armature 550 with minimal slack and play, but without binding as it is slid along the length of the armature by the gloved hands 2242 of the practitioner 2240. The practitioner takes care to touch only the unfurling drape as it is slid along the armature, thus maintaining a sterile environment. Significantly, the act of draping the probe, holder and armature can be performed easily by a single person, as the shape of the support device remains fairly rigid during the draping procedure. In fact, the draping can even be accomplished with one hand if needed.

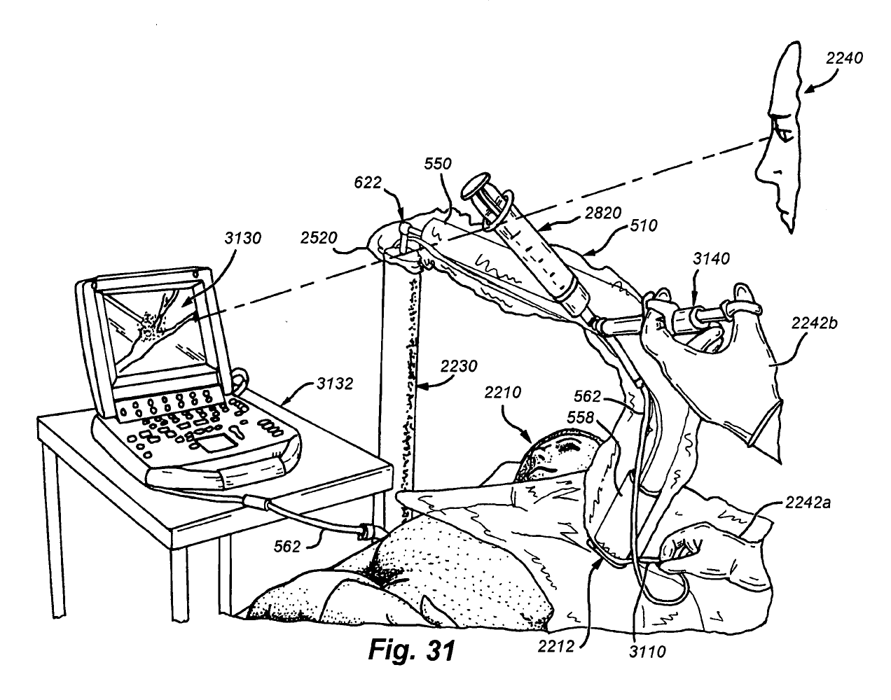

According to FIG. 25, the probe 560, holder 558, armature 550, ball joint assembly 622 and at least a portion of the stand 2230 are now fully draped to create the requisite sterile area around the patient. The probe lead 562 is shown exiting from the proximal end 2520 of the drape 2310 to interconnect with a remote imaging system (see FIG. 31) within the convenient view of the practitioner 2240. In alternate embodiments, described above, the lead can be carried internally of the support device 510 and/or directly connected to the system without exposed leads. The distal end 2530 of the drape 2310 is drawn snugly against the tip of the probe 560 so that it will not interfere with the probe's transmitted or received scanning signal relative to the region 2212.

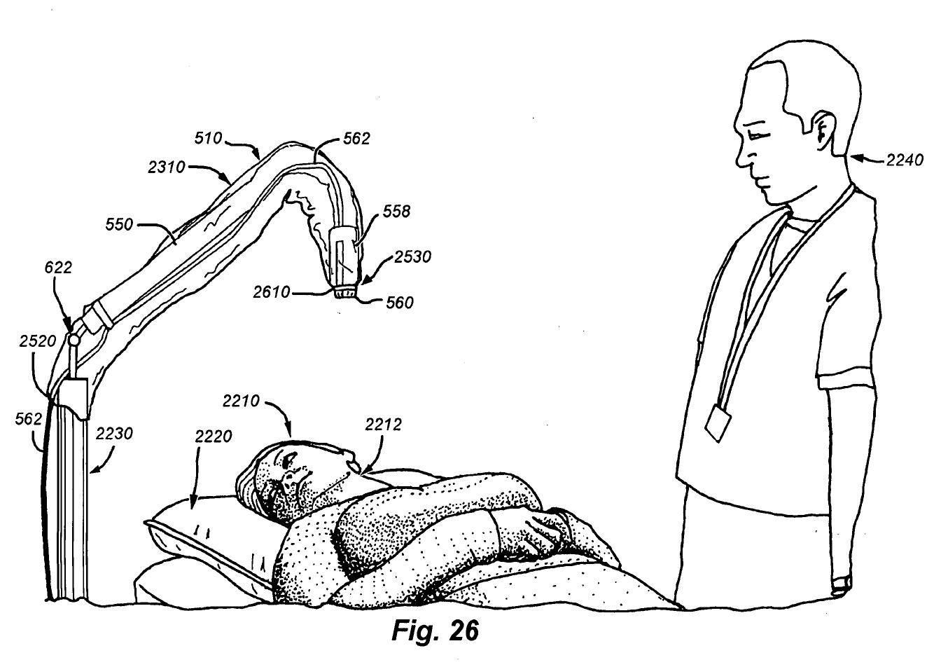

In the next step, shown in FIG. 26, the distal end 2530 of the drape 2310 is maintained tautly in contact with the probe tip using a sterile elastic band 2610. The band is applied after the armature 510 has been flexed upwardly and the ball joint assembly 622 has been pivotally raised to afford the practitioner 2240 extra room to work. The ball joint may include a friction brake (not shown) to hold the armature in the raised position.

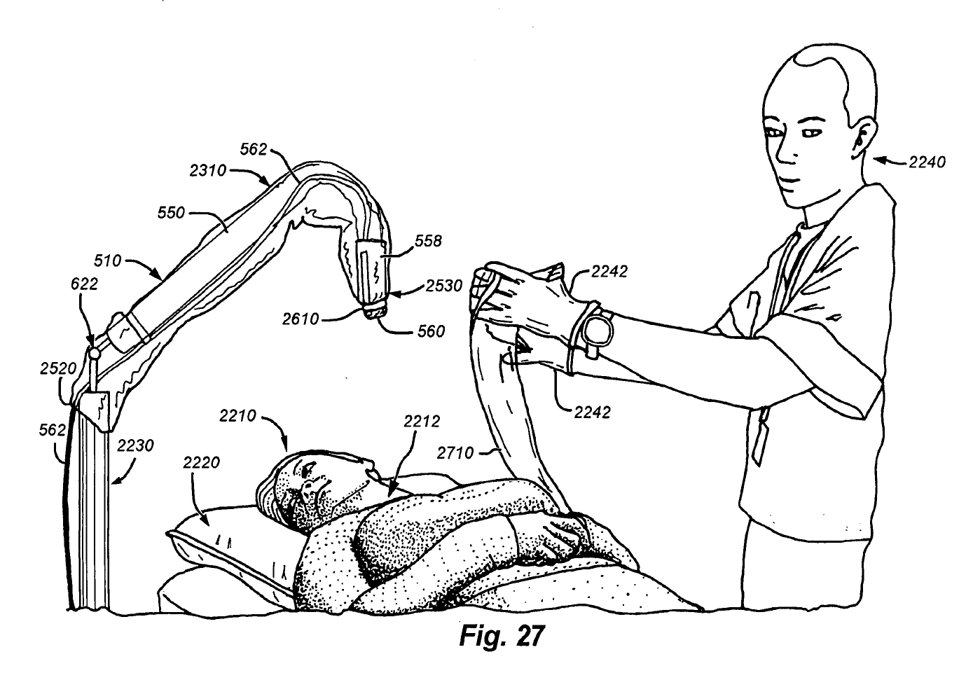

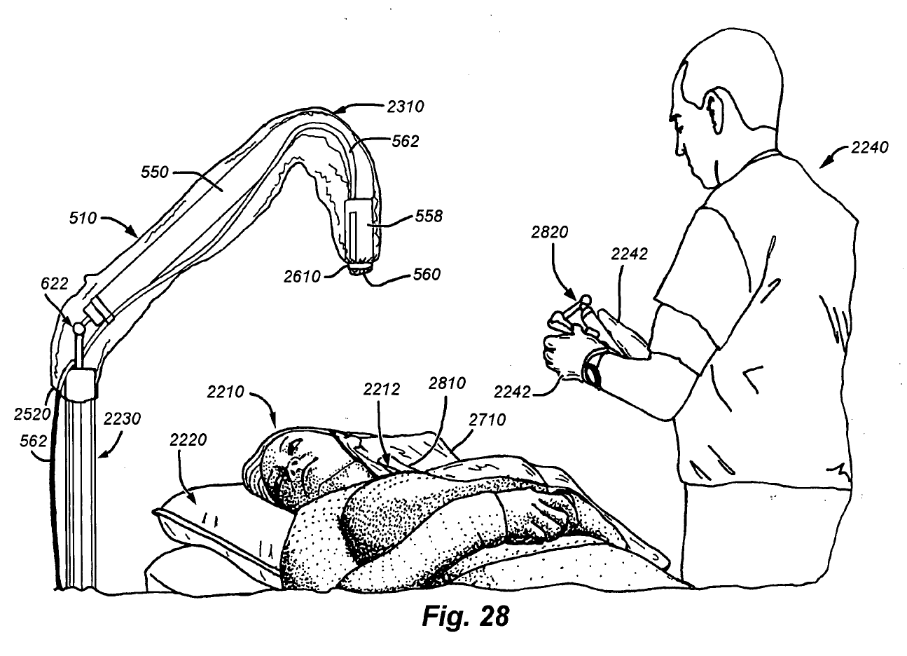

In FIG. 27, the support device 510 remains raised as described (referencing FIG. 26 above) so that the practitioner 2240 can apply a sterile polymeric drape 2710 to the region of interest 2212. The drape includes an aperture (see FIG. 28 below) that is centered about the region of interest/treatment 2212. This draping step can be omitted in certain procedures, but serves to better create a sterile field of operation.

In the next step of the procedure, illustrated in FIG. 28, the practitioner has centered the drape 2710 with respect to the region of interest/treatment 2212. In this manner, the drape's aperture 2810 exposes the region of interest 2212 while the surrounding skin and clothing of the patient is covered by the sterile drape. Adhesive strips (not shown) can be applied to maintain the drape 2710 in place if desired. The practitioner 2240, at this time, prepares the anesthetic delivery system 2820 for insertion of the system's needle.

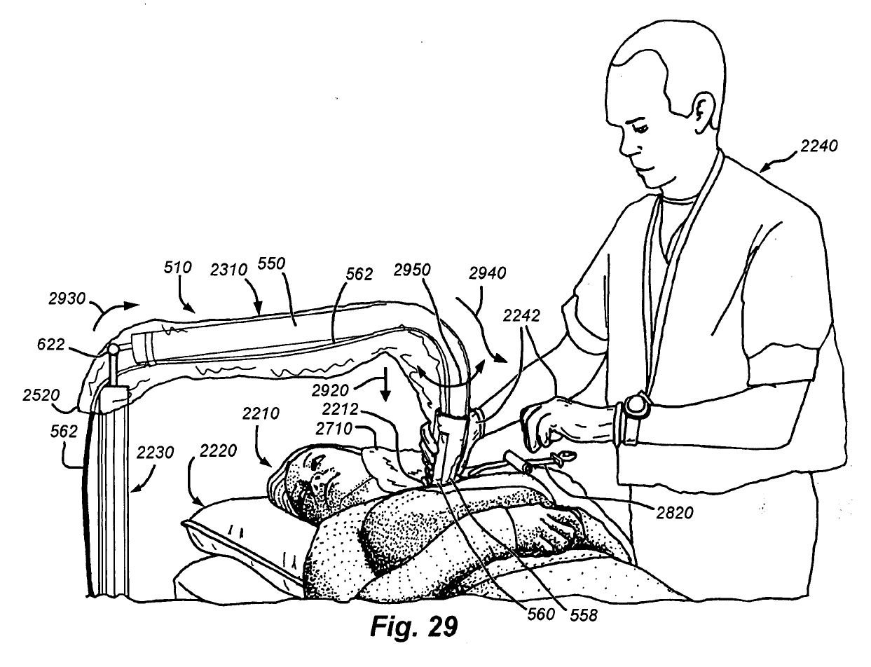

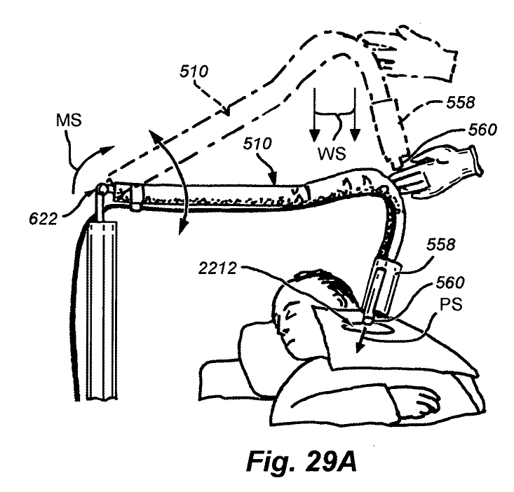

In FIG. 29, the practitioner 2240 has introduced the needle to the region of interest/treatment 2212, and is preparing to guide the needle to the subcutaneous target with the aid of the imaging system via the probe 560. As such, the practitioner 2240 grasps the handle of the probe and reorients it with predetermined force into engagement with patient's region of interest/treatment 2212. In this example, the practitioner 2240 moves the armature as a unit downwardly toward the region 2212 by applying a downforce (arrow 2920) to the draped holder 558 that pivots (curved arrow 2930) the ball joint assembly 622. To accurately orient the angle-of-attack and precise position of the probe tip on the region 2212, the practitioner also flexes (curvaceous arrow 2940) the armature 550 to reorient it as shown. To attain the desired rotation of the tip, the practitioner simultaneously rotates (curved double arrow 2950) the holder 558, which propagates rotation through the armature structure in this embodiment to attain the desired final rotational orientation.

As discussed above, the armature can be provided with a segment-to-segment locking mechanism. In one embodiment, the practitioner releases the normally active locking mechanism via a switch or mechanical linkage during movement and relocks the armature when a desired orientation is achieved. In another embodiment, the armature remains unlocked until the practitioner positively applies the locking function after desired orientation. In either case, the final position, as shown in FIG. 29 provides a transmitted image from the probe 560 that is sufficient to assist the practitioner 2240 in guiding the needle to its target site. The armature can be locked (in some embodiments) when such an image is acquired. Note, in other embodiments, the friction normally exerted between segments may be sufficient to maintain the probe on station without application of further locking action.

As described above, the final position of the probe engaging the region of interest/treatment 2212 is maintained in part by the application of device-weight-induced pressure to the region. This pressure is derived from the relatively loose joint of the ball joint assembly 622, which allows a predetermined amount of play in the overall structure. As shown in FIG. 29A, the weight WS of the support device 510 generates a moment MS in the ball joint assembly 622. The weight creates a moderate downforce so that, when the probe is brought into relatively close proximity to the surface, the desired pressure PS against the region 2212 is generated. This pressure PS is sufficient to maintain contact, but not so excessive as to cause dislocation or distortion of internal structures, which would obscure the acquired image and degrade the ability to guide the needle. In one embodiment the pressure is in a range of between approximately 0.05 and 1.2 psi.

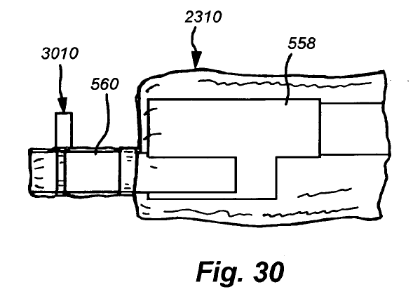

As shown in FIG. 30, the practitioner may opt to mount a sterile polymeric or metallic needle guide 3010 on the probe 560 to assist in the insertion of the needle at the appropriate location with respect to the probe's viewing/scanning area.

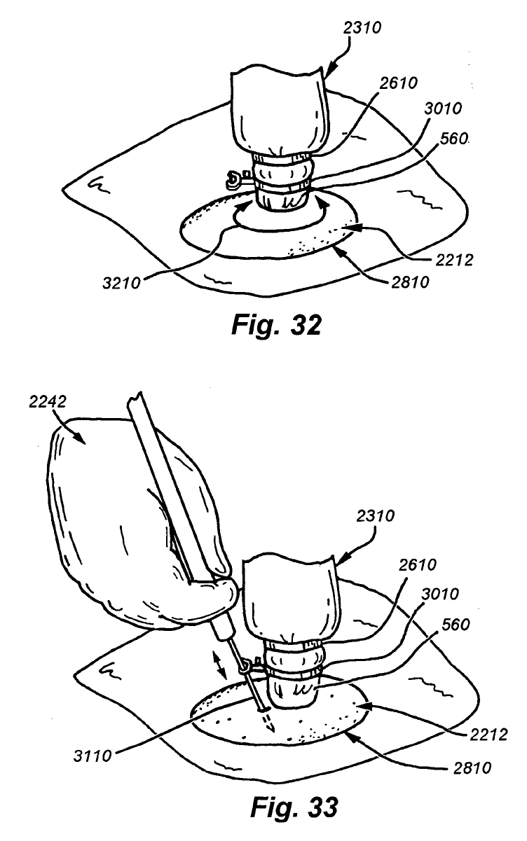

Referring now to FIG. 31, having properly applied the probe and allowing the support device 510 to maintain it accurately with respect to the region of interest/treatment 2212, the practitioner 2040 can use one hand 2242a to guide the needle 3110 to its target site while maintaining a clear image on the region in the viewed display 3130 of an interconnected imaging system 3132. The practitioner's other hand 2242b is free to operate a syringe 3140 to inject the medicament via the needle 3110, or perform another function, as needed. No hand is required to maintain the probe so long as the region remains reasonably stationary during this phase of the procedure. This outcome stands in clear contrast to the prior art approach in which a second practitioner must continually engage and reorient the image to serve the viewing needs of the first practitioner as he or she inserts the needle and injects the medicament.

Where final adjustment of the probe or reorientation (or particularly, rotation of the probe) is required, such a function is accomplished quickly and easily. As detailed in FIG. 32, the probe 560 is rotated (curved double arrow 3210) while unlocked to achieve optimum positioning. This is confirmed by viewing the resulting image in the display 3130. Once correct rotation is attained or reestablished, the probe can be unhanded, and the practitioner's hand 2242 is free to insert or reinsert the needle 3110 via the needle guide 3010 or via a separate guide, or using freehand techniques. The insertion and guiding are monitored using the display 3130 until the target is reached. As stated, rotation is readily achieved with the armature of this invention as a large number of adjacent segments can absorb an incremental amount of a total rotation. Alternatively a frictional wrist at the holder can be employed to absorb most or all of the rotation.

H. Probe Attachment Mechanisms

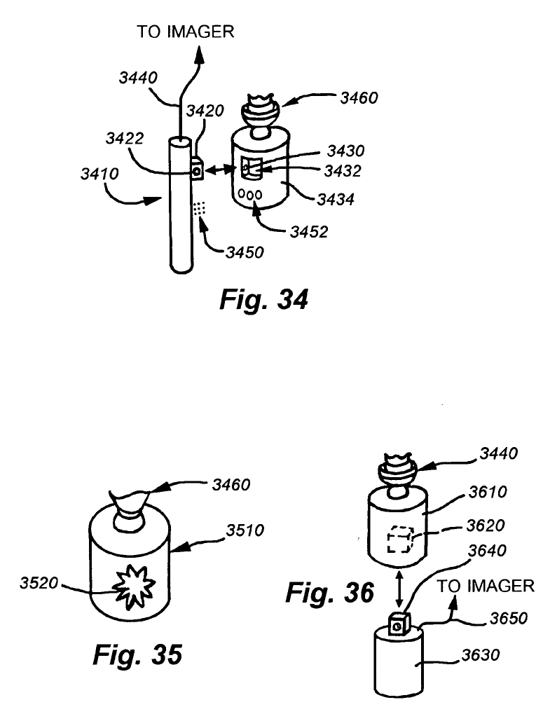

As described above, the probe can be mounted to the armature via a holder that firmly supports the probe at a desired angle. In alternate embodiments, the probe can be attached using a quick-disconnect mechanism in which the housing of the probe is custom-designed or modified to include a direct-attachment structure. As shown in the embodiment of FIG. 34, the probe 3410 includes a body-mounted clip 3420 with one or more spring-loaded balls 3422 to removably engage corresponding detents 3430 in a confirming aperture 3432 of a distal holder/mounting 3434. The probe can include an external wire lead 3440 interconnected to the imager, or it can include an integral connector 3450 is (shown in phantom) that engages a corresponding connector 3452 (also shown in phantom) on the holder/mounting 3434. The leads for this integrated connector are typically routed through or as part of the internal'structure of the armature 3460.

The aperture shape is highly variable as illustrated by the holder/mounting 3510 of FIG. 35. In this embodiment, the aperture 3520 defines a star pattern. A variety of shapes can be provided. In the case of the star pattern, or another regular polygon with more than four sides, a larger number of angular orientations of the probe with respect to the holder can be provided.

Finally, FIG. 36 shows a holder/mounting 3610 with an axially positioned aperture 3620. The aperture 3620 removably receives a “snap-in” probe 3630 with a conforming projection 3640. The probe in this example includes an external imager lead 3650. In alternate embodiments, an internal connector structure, routed through the armature 3460 can be provided. Likewise, the shape of the aperture 3620 and conforming projection 3640 is highly variable. In further embodiments, the probe can include a more-positive locking mechanism with respect to the holder that must be released by the practitioner to allow removal of the probe.

The foregoing has been a detailed description of illustrative embodiments of the invention. Various modifications and additions can be made without departing from the spirit and scope thereof. For example, it is expressly contemplated that the components employed for the holder, armature and/or other parts of the inventive system can be constructed from materials that are generally transparent to X-ray radiation and other modalities for deep scanning of tissue. Conversely, some components can be adapted to fluoresce in response to scanning radiation for improved control of positioning and other tracking purposes. The arrangement of mounting structures and holders relative to the flexible armature are highly variable. In addition the degree to which individual segments can bend/flex relative to adjacent segments is also highly variable. The normal level of unlocked friction between segments may vary. Locking of segments or groups of segments may or may not be provided in various embodiments. Also, while thumbscrews are employed for a variety of locking functions, it should be clear to those of ordinary skill that alternative locking arrangements can be provided to various components, such as hoop-stress-applying bands, movable pressure plates, ratchet and pawl systems and locking gear trains. Similarly, mechanical or other means for preventing overextension of the segmented or flexible joints may be included. Another embodiment may include controls for the imaging or other medical device on the probe holder where they are easily accessible by the practitioner user. Furthermore, the use of biocompatible materials as known by anyone of skill for all subassemblies described or referred to herein are incorporated and encouraged. Accordingly, this description is meant to be taken only by way of example, and not to otherwise limit the scope of the invention.

What is claimed is:

1. A system for supporting medical instruments with respect to a region of interest on a patient comprising:
a base that interconnects to a substantially stationary structure, the base interconnected with a proximal end of a flexible armature section, the flexible armature section comprising a plurality of discrete polymer segments each defining an inner lumen and including a hemispherical nose section adapted to interengage a tail section and thereby afford a predetermined degree of bending and rotation therebetween;
an instrument holder interconnected with a distal end of the flexible armature and removably attaching an instrument thereto; and
a selective actuatable lock that applies increased frictional pressure between the nose section and the tail section to thereby lock a portion of the flexible armature section.

2. The system as set forth in claim 1 wherein the lock comprises a hoop-stress generating assembly that applies a hoop stress to the tail section so as to forcibly engage the nose section.

3. The system as set forth in claim 1 wherein the lock comprises a tension cable attached adjacent to each of the proximal end and the distal end and that passes through a bulkhead in each segment and that is adapted to be selectively tensioned to axially compress the nose with respect to the tail section.

4. The system as set forth in claim 3 wherein each bulkhead includes a passage for a probe lead to pass therethrough.

5. The system as set forth in claim 3 wherein the holder includes a tensioning assembly that tensions the cable.

6. The system as set forth in claim 5 wherein the tensioning assembly comprises a base fixed to the cable and a hand-rotated member threadingly attached to the base.

7. The system as set forth in claim 1 wherein the lock comprises a vacuum assembly that selectively creates a relative vacuum in a sealed space within the armature section between the proximal end and the distal end.

8. The system as set forth in claim 7 further comprising a conduit that passes through the sealed space adapted to direct leads therethrough from the proximal end to the distal end in isolation from the sealed space.

9. A system for supporting medical instruments with respect to a region of interest on a patient comprising:
a base that interconnects to a substantially stationary structure, the base interconnected with a proximal end of a flexible armature section, the flexible armature section comprising a plurality of discrete segments each defining an inner lumen and including a hemispherical nose section adapted to interengage a tail section and thereby afford a predetermined degree of bending and rotation therebetween;
an instrument holder interconnected with a distal end of the flexible armature and removably attaching an instrument thereto; and
wherein the base includes a pivoting joint assembly that allows the armature section and the instrument holder to float with respect to the region of interest and apply a predetermined weight-generated pressure upon the region of interest.

10. The system as set forth in claim 9 wherein the joint comprises a ball joint and includes a stem adapted to rotate about a predetermined axis transverse to an axis of rotation of the pivoting joint.

11. The system as set forth in claim 10 further comprising a lock that fixes rotation about the predetermined axis.

12. The system as set forth in claim 9 wherein the medical instrument comprises a probe operatively interconnected to an electronic device that receives a signal from the probe and the base is mechanically connected to a housing of the electronic device.

13. The system as set forth in claim 12 wherein the electronic device comprises a hand-portable ultrasound device and the probe comprises an ultrasound probe.

14. The system as set forth in claim 9 wherein the base includes a clamp adapted to removably attach to at least one of a bed rail, a ceiling, an IV drug stand and a wall plate.

15. The system as set forth in claim 9 wherein the instrument holder includes an elastomeric pad assembly adapted to resiliently and removably engage a plurality of shapes of medical instrument.

16. A method for guiding a medical instrument into a subcutaneous area beneath a skin region of a patient, comprising the steps of:
supporting an ultrasound probe, the probe being interconnected to an ultrasound display device within view, in a holder at a distal end of a flexible armature section, the flexible armature section comprising a plurality of discrete polymer segments each defining an inner lumen and including a hemispherical nose section adapted to interengage a tail section and thereby afford a predetermined degree of bending and rotation therebetween;
draping the probe and armature section with a sterile drape that is opened at a proximal end to slide over the armature and sealed at a distal end placed in engagement with a tip of the probe;
reorienting the tip of the ultrasound probe into engagement with the skin region by flexing the armature to overcome predetermined friction between the interengaged nose section and tail section of at least some of the segments to bend and rotate the at least some segments with respect to interengaged others of the segments so as to acquire a desired image at the display device, the armature section maintaining a predetermined reoriented shape at least by the predetermined friction; and
guiding a needle to a subcutaneous target with at least one hand while viewing the image and while the ultrasound probe and the instrument holder remains ungrasped by another hand.

17. The method as set forth in claim 16 wherein the steps of draping, reorienting and guiding are each performed by at least one hand of the same discrete practitioner.

18. The method as set forth in claim 17 further comprising locking the flexible armature in place after reorienting.

19. The method as set forth in claim 16 further comprising engaging the skin region with a weight-generated force applied due to pivotal movement of a floating joint operatively connected to a proximal end of the flexible armature.

20. The method as set forth in claim 19 further comprising attaching the joint to a base mounted on the display device.
